(19) United States
(12) Patent Application Publication
(10) Pub. No.: US 2018/0245121 A1
(43) Pub. Date: Aug. 30, 2018

(54) ELECTROCHEMICAL BIOSENSOR

(71) Applicant: THE UNIVERSITY OF QUEENSLAND, Queensland (AU)

(72) Inventors: Kirill ALEXANDROV, Queensland (AU); Viktor STEIN, Darmstadt (DE); Zhong GUO, Queensland (AU)

(73) Assignee: THE UNIVERSITY OF QUEENSLAND, Queensland (AU)

(21) Appl. No.: 15/578,570

(22) PCT Filed: Jun. 1, 2016
(86) PCT No.: PCT/AU2016/050436

§ 371 Date: Nov. 30, 2017

(30) Foreign Application Priority Data
Jun. 1, 2015 (AU) 2015902093

(51) Int. Cl. C12Q 1/00; C12Q 1/32; C12Q 1/37; C12N 9/04; C12N 15/63

(52) U.S. Cl. CPC C12Q 1/005; C12Q 1/32; C12Q 1/37; C12Y 101/9901; C12N 15/63; C12Q 2565/607; C12N 9/0006

(57) ABSTRACT

A biosensor comprises an amino acid sequence of an enzyme such as glucose dehydrogenase which is capable of reacting with a substrate to produce one or more electrons, wherein the enzyme has been engineered to be switchable from a catalytically inactive to a catalytically active state in response to binding a target molecule. A method of detecting a target molecule is provided wherein an enzyme such as glucose dehydrogenase reacts with a substrate to produce one or more electrons as a result of the enzyme switching from a catalytically inactive to a catalytically active state in response to binding the target molecule.

### TECHNICAL FIELD

THIS INVENTION relates to biosensors. More particularly, this invention relates to an electrochemical biosensor and to electrochemically active enzymes or fragments thereof that are suitable for detection of one or more target molecules in a sample. The biosensor molecule may also relate to the field of synthetic biology such as for constructing artificial cellular signalling networks.

### BACKGROUND

Detection of target molecules or analytes in biological samples is central to diagnostic monitoring of health and disease. Key requirements of analyte detection are specificity and sensitivity, particularly when the target molecule or analyte is in a limiting amount or concentration in a biological sample.

Typically, specificity is provided by monoclonal antibodies which specifically bind the analyte. Sensitivity is typically provided by a label bound to the specific antibody, or to a secondary antibody which assists detection of relatively low levels of analyte. This type of diagnostic approach has become well known and widely used in the enzyme-linked immunosorbent sandwich assay (ELISA) format. In some cases, enzyme amplification can even further improve sensitivity such as by using a product of a proenzyme cleavage reaction catalyzing the same reaction. Some examples of such “autocatalytic” enzymes are trypsinogen, pepsinogen, or the blood coagulation factor XII. However, in relation to specificity antibodies are relatively expensive and can be difficult to produce with sufficient specificity for some analytes. Polyclonal antibodies also suffer from the same shortcomings and are even more difficult to produce and purify on a large scale.

Current methods to detect specific target molecules and analytes for either prognostic or diagnostic purposes suffer from a number of limitations which significantly restrict their widespread application in clinical, peri-operative and point-of-care settings. Most importantly, the vast majority of diagnostic assays require a significant level of technical expertise and a panel of expensive and specific reagents (most notably monoclonal antibodies) along with elaborate biomedical infrastructures which are rarely available outside specialized laboratory environments. For instance, ELISAs—the gold standard for detecting specific analytes in complex biological samples—rely on the selective capture of a target analyte on a solid surface which in turn is detected with a second affinity reagent that is specific for the target analyte. ELISAs also feature extensive incubation and washing steps which are generally time consuming and difficult to standardize as the number of successive steps frequently introduces significant variation across different procedures, operators and laboratories making quantitative comparisons difficult.

### SUMMARY

The present invention addresses a need to develop quantitative, relatively inexpensive and easily produced molecular biosensors that readily detect the presence or the activity of target molecules (e.g analytes) on short time scales that are compatible with treatment regimes. Such biosensors can either be applied singly or in multiplex to validate and/or diagnose molecular phenotypes with high specificity and great statistical confidence irrespective of the genetic background and natural variations in unrelated physiological processes. Such molecular biosensors may be used in other testing procedures such as where the target molecule or analyte is an illicit drug or performance-enhancing substance.

More particularly, the present invention provides a molecular biosensor that is particularly suited to incorporation into electrical devices such as point-of-care devices for analysis and transmission of diagnostic results.

It is therefore an object of the invention to provide a biosensor molecule which has specificity for a target molecule and which can produce an electrical response to detection of the target molecule.

In one broad form the invention relates to a biosensor comprising an amino acid sequence of an enzyme which is capable of reacting with a substrate to produce one or more electrons, wherein the enzyme has been engineered to be switchable from a catalytically inactive to a catalytically active state in response to binding a target molecule.

In a first aspect, the invention provides a biosensor comprising at least one amino acid sequence of an enzyme capable of reacting with a substrate molecule when in a catalytically active state to produce one or more electrons; and at least one heterologous, sensor amino acid sequence that releasably maintains the enzyme in a catalytically inactive state, wherein the heterologous, sensor amino acid sequence is responsive to a target molecule to switch the amino acid sequence of the enzyme from the catalytically inactive state to said catalytically active state. Thus, the heterologous, sensor amino acid sequence reversibly regulates catalytic activity of the enzyme. The heterologous, sensor amino acid sequence can be displaced in the presence of the target molecule to thereby catalytically activate the enzyme. The heterologous, sensor amino acid sequence can allosterically regulate the catalytic activity of the enzyme.

Suitably, the at least one enzyme amino acid sequence and said at least one heterologous, sensor amino acid sequence are present in, or form at least part of a single, contiguous amino acid sequence.

In a particular embodiment, said at least one heterologous, sensor amino acid sequence is an insert in said at least one enzyme amino acid sequence, to thereby facilitate switching the enzyme amino acid sequence between said catalytically inactive and said catalytically active state.

Suitably, the heterologous, sensor amino acid sequence binds said target molecule to thereby switch the amino acid sequence of the enzyme from the catalytically inactive state to said catalytically active state.

In one embodiment, the heterologous, sensor amino acid sequence is an amino acid sequence of a calcium-binding protein, or a fragment thereof. In a particular embodiment, the calcium-binding protein is calmodulin.

In a second aspect, the invention provides a biosensor comprising at least one amino acid sequence of an enzyme capable of reacting with a substrate molecule when in a catalytically active state to produce one or more electrons; and at least one other amino acid sequence of said enzyme which is engineered to releasably maintain the enzyme in a catalytically inactive state, wherein the biosensor is responsive to a target molecule to switch the amino acid sequence of the enzyme from the catalytically inactive state to said catalytically active state.

Suitably, said at least one other amino acid sequence of said enzyme is engineered to comprise one or more amino acid sequence mutations. Suitably, said at least one amino acid sequence of the enzyme capable of reacting with a substrate molecule when in a catalytically active state to produce one or more electrons and said at least one other amino acid sequence of said enzyme engineered to releasably maintain the enzyme in a catalytically inactive state, non-covalently interact. In one embodiment, the biosensor of this aspect further comprises yet another amino acid sequence of said enzyme which is capable of replacing said at least one other amino acid sequence of said enzyme engineered to releasably maintain the enzyme in a catalytically inactive state. Typically, this replacement restores the catalytic activity of the enzyme by non-covalently combining said yet another amino acid sequence of said enzyme with said at least one amino acid sequence of the enzyme capable of reacting with a substrate to form a functional, catalytically active enzyme. In a broad embodiment, said yet another amino acid sequence of said enzyme and said at least one amino acid sequence of the enzyme capable of reacting with a substrate comprise respective binding moieties that can interact, such as by binding a target molecule, to facilitate the replacement of the engineered amino acid sequence by said yet another amino acid sequence.

A particular embodiment of the second aspect therefore provides a biosensor comprising a first component that comprises: at least one amino acid sequence of an enzyme capable of reacting with a substrate molecule when in a catalytically active state to produce one or more electrons; a first binding moiety; and at least one other amino acid sequence of said enzyme which is engineered to releasably maintain the enzyme in a catalytically inactive state; and a second component comprising at least one other amino acid sequence of said enzyme and a second binding moiety; arranged so that an interaction between said first and second binding moieties facilitates replacement of said at least one other amino acid sequence of the first component by said at least one other amino acid sequence of the second component, to thereby switch the enzyme of the first component from a catalytically inactive state to a catalytically active state.

In another broad embodiment, said engineered amino acid sequence of said enzyme and said at least one amino acid sequence of the enzyme capable of reacting with a substrate comprise respective binding moieties that initially interact, which interaction is subsequently disrupted by one or the other of the binding moieties binding a target molecule. This disruption of the interaction facilitates the replacement of the engineered amino acid sequence by said yet another amino acid sequence.

Another particular embodiment of the second aspect therefore provides a biosensor comprising a first component comprising: at least one an amino acid sequence of an enzyme capable of reacting with a substrate molecule when in a catalytically active state to produce one or more electrons; a first binding moiety; and at least one other amino acid sequence of said enzyme which is engineered to releasably maintain the enzyme in a catalytically inactive state; and a second component comprising at least one other amino acid sequence of said enzyme and a second binding moiety; arranged so that an interaction between said first and second binding moieties is released by a target molecule capable of binding the first or second binding moiety to facilitate replacement of said at least one other amino acid sequence of the first molecule by said at least one other amino acid sequence of the second molecule, to thereby switch the enzyme of the first component from a catalytically inactive state to a catalytically active state.

In another broad embodiment, the biosensor of the second aspect is suitable for detecting a protease target molecule and thus typically comprises one or more, such as two or three protease cleavage sites. Preferably, the one or more protease cleavage sites are located said yet another amino acid sequence of the enzyme capable of replacing the engineered amino acid sequence. Said yet another amino acid sequence may further comprise a sequence enhancing binding and/or cleavage efficiency of the protease, which may be located proximally to the protease cleavage site.

Preferably, said at least one amino acid sequence of the enzyme capable of reacting with a substrate and said yet another amino acid sequence of the enzyme comprise respective binding moieties that can interact after protease cleavage of an inhibitor of binding between these. This interaction facilitates the replacement of the engineered amino acid sequence by said yet another amino acid sequence.

Yet another particular embodiment of the second aspect therefore provides a biosensor comprising a first component comprising: at least one an amino acid sequence of an enzyme capable of reacting with a substrate molecule when in a catalytically active state to produce one or more electrons; a first binding moiety; and at least one other amino acid sequence of said enzyme which is engineered to releasably maintain the enzyme in a catalytically inactive state; and a second component comprising at least one other amino acid sequence of said enzyme and a second binding moiety linked or connected to an inhibitor by a protease cleavage site, wherein the inhibitor prevents or inhibits an interaction between the first and second binding moieties; arranged so that said inhibitor is released by a protease target molecule cleaving said protease cleavage site to facilitate an interaction between the first and second binding moieties to facilitate replacement of said at least one other amino acid sequence of the first molecule by said at least one other amino acid sequence of the second molecule, to thereby switch the enzyme of the first component from a catalytically inactive state to a catalytically active state.

In a particular embodiment, the inhibitor is substantially the same molecule as the first binding moiety.

In a third aspect, the invention provides a biosensor comprising at least one amino acid sequence of an enzyme capable of reacting with a substrate molecule when in a catalytically active state to produce one or more electrons; a binding moiety capable of binding a target molecule; and at least one enzyme inhibitor which is capable of interacting with the binding moiety in the absence of the target molecule to thereby inhibit the enzyme; arranged so that the target molecule can release the interaction between said at least one enzyme inhibitor and the binding moiety to thereby release inhibition of the enzyme by the inhibitor and switch the amino acid sequence of the enzyme from a catalytically inactive state to said catalytically active state.

An embodiment of the third aspect provides a biosensor comprising a first component comprising: at least one amino acid sequence of an enzyme capable of reacting with a substrate molecule when in a catalytically active state to produce one or more electrons; an inhibitor of said enzyme linked or coupled to the enzyme by a protease cleavage site; and a first component binding moiety; a second component comprising a second component binding moiety capable of binding the first component binding moiety; a protease amino acid sequence; and another second component binding moiety capable of binding a target molecule; and a third component comprising a third component binding moiety that can interact with said second component binding moieties in the absence of the target molecule; arranged so that said target molecule can displace binding between the third component binding moiety and said second component binding moieties to facilitate an interaction between said first component binding moiety and said second component binding moiety whereby the protease cleaves the protease cleavage site to remove inhibition of the enzyme by the inhibitor and thereby switch the enzyme from a catalytically inactive state to a catalytically active state.

In all of the above aspects the enzyme may be an oxidoreductase enzyme, preferably a glucose dehydrogenase enzyme.

The invention further provides an oxidoreductase enzyme, preferably a glucose dehydrogenase (GDH) enzyme, comprising a heterologous, sensor amino acid sequence which is responsive to a target molecule, wherein binding of the target molecule acts to regulate catalytic activity of the enzyme.

The invention also provides an oxidoreductase enzyme, preferably a glucose dehydrogenase (GDH) enzyme, comprising an inhibitory moiety acting to prevent or reduce catalytic activity of the enzyme, wherein the inhibitory moiety can be displaced in the presence of one or more molecules to activate catalytic activity of the enzyme. The invention further provides a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme. Another aspect of the invention provides a composition or kit comprising the biosensor, oxidoreductase enzyme, GDH enzyme, or the polypeptides comprising first and second fragment sequences of a GDH enzyme of any of the aforementioned aspects The composition or kit may further comprise a substrate molecule.

A further aspect of the invention provides a method of detecting a target molecule, said method including the step of contacting the biosensor, oxidoreductase or GDH enzyme or polypeptides comprising first and second fragment sequences of a GDH enzyme of any of the aforementioned aspects with a sample to thereby determine the presence or absence of the target molecule in the sample.

A yet further aspect of the invention provides a method of diagnosis of a disease or condition in an organism, said method including the step of contacting the biosensor, oxidoreductase or GDH enzyme or polypeptides comprising first and second fragment sequences of a GDH enzyme of any of the aforementioned aspects with a biological sample obtained from the organism to thereby determine the presence or absence of a target molecule in the biological sample, determination of the presence or absence of the target molecule facilitating diagnosis of the disease or condition.

The organism may include plants and animals inclusive of fish, avians and mammals such as humans.

A still yet further aspect of the invention provides a detection device that comprises a cell or chamber that comprises the biosensor, oxidoreductase or GDH enzyme or polypeptides comprising first and second fragment sequences of a GDH enzyme of any of the aforementioned aspects.

Suitably, a sample may be introduced into the cell or chamber to thereby facilitate detection of a target molecule.

In certain embodiments, the detection device is capable of providing an electrochemical, acoustic and/or optical signal that indicates the presence of the target molecule.

The detection device may further provide a disease diagnosis from a diagnostic target result by comprising:
a processor; and
a memory coupled to the processor, the memory including computer readable program code components that, when executed by the processor, perform a set of functions including:
analysing a diagnostic test result and providing a diagnosis of
the disease or condition.

The detection device may further provide for communicating a diagnostic test result by comprising:
a processor; and
a memory coupled to the processor, the memory including computer readable program code components that, when executed by the processor, perform a set of functions including:
transmitting a diagnostic result to a receiving device; and
optionally receiving a diagnosis of the disease or condition from the or another receiving device.

A related aspect of the invention provides an isolated nucleic acid encoding the biosensor of any of the aforementioned aspects, or a component thereof, or an oxidoreductase enzyme or GDH enzyme of the invention or a polypeptide comprising a first or second fragment sequence of a GDH enzyme of the invention.

Another related aspect of the invention provides a genetic construct comprising the isolated nucleic acid of the aforementioned aspect.

A further related aspect of the invention provides a host cell comprising the genetic construct of the aforementioned aspect.

A still further related aspect provides a method of producing a recombinant protein biosensor or a component thereof or an oxidoreductase enzyme or GDH enzyme of the invention or a polypeptide comprising a first or second fragment sequence of a GDH enzyme, said method including the step of producing the recombinant protein biosensor or a component thereof in the host cell of the previous aspect.

### BRIEF DESCRIPTION OF THE FIGURES

FIG. 1. Structure of A. calcoaceticus PQQ-GDH and identification of calmodulin insertion site. (A) Ribbon representation of the enzyme in complex with PQQ and glucose. The PQQ cofactor is displayed in ball and stick representation while glucose is colored in atomic colors. The bound Ca²⁺ is displayed as space filing object. The β-sheets are marked with respective numbers and the β-strands of the sheet 3 and marked by letters. The strands 3 A and 3B are colored in blue and the active site residues involved in coordination of glucose are displayed in ball and stick. The catalytic His144 is colored in red. (B) the side view of GDH displaying the loop connecting strands A and B. The structure displayed and colored as in A.

FIG. 2. Spectrometric analysis of PQQ-GDH-CaM activity at different concentrations of Ca²⁺. (A) Time resolved changes in absorption of 60 μM electron accepting dye dichlorophenolindophenol in the presence of 0.6 mM electron mediator phenazine methosulphate were measured at 600 nm in the presence of 20 mM of glucose and 1 nM GDH-CaM. (B) As in A, but using 3 nM GDH-CaM exposed to the increasing concentrations of CaCl₂. (C) Performance of GDH-CaM chimer as a sensor in electrochemical systems. Main plot; response of GDH-CaM chronoamperometric electrode to increasing Ca2+ concentrations. Current measured after 5 s at +0.4V versus imbedded Ag reference strip, GDH-CaM present at 300 nM, PMS mediator at 3 mM, glucose at 50 mM. Inset plot; current versus time after polarization at +0.4V versus imbedded Ag reference strip at two representative calcium concentrations (0 and 100 μM). (D) A plot of the observed initial reaction rates of 3 nM GDH-CaM in the presence of 20 mM glucose and 1.1 mM of the specific Ca2+ chelator BAPTA. The experiments were performed as in (B). Filled triangles represent experiments in which titration was performed in the presence of 2 mM MgCl₂.

FIG. 3. (A) Pyrroloquinoline Quinone Glucose Dehydrogenase linked with inhibitory peptide NSTHHHHFATIW (SEQ ID NO: 51) described by Abe K, et al. (2013) via a protease cleavable linker. In the assay, absorption of 10 μM electron accepting dye dichlorophenolindophenol in the presence of 0.3 mM electron mediator phenazine methosulphate were measured at 600 nm in the presence of 20 mM of glucose, 50 uM CaCl₂ and 2 nM GDH-AI. 10 uM of TVMV protease were used. (B) Pyrroloquinoline Quinone Glucose Dehydrogenase linked via thrombin cleavable linker with the inhibitory peptide SHDIHYM identified by phage display. Activity was measured as in (A), using 60 μM of dichlorophenolindophenol, 0.6 mM of phenazine methosulphate, 20 mM of glucose, 50 uM CaCl₂, 5 nM GDH-AI and 20 U of Thrombin were used in the assay. (C) Schematic representation of a biosensor based on Pyrroloquinoline Quinone Glucose Dehydrogenase C-terminally fused to an inhibitory antibody fragment through a TVMV cleavable linker. (D) activity analysis of several biosensors carrying different antibody domains. Activity was measured as in (B). (E) schematic representation of a two component receptor architecture where a active or autoinhibited version of a protease is brought into proximity if auto inhibited GDH by analyte-mediated scaffolding interactions (F) rapamycin-mediated activation of the two component receptor depicted in E. In this assay, 1 nM GDH-VH-FKBP12, 15 nM FRB-TVMV were incubated with 60 μM of dichlorophenolindophenol, 0.6 mM of phenazine methosulphate, 50 uM CaCl₂ in the presence and in the absence of 100 nM Rapamycin for 90 min. Absorption at 600 nM was monitored after adding 20 mM of glucose. (G) schematic representation of a reversible two component biosensor architecture based on the auto inhibited GDH. In this architecture the inhibitory domain is fused to a ligand peptide such as calmodulin binding peptide or the affinity clamp ligand peptide. The second component of the system is represented by the binding domain such as calmodulin or the affinity clamp binding peptide. Scaffolding of both molecules by the ligand results in “tag of war” interaction of the AI with GDH and fused peptide with its binding domain resulting in enzyme's activation. (H) Schematic representation of a biosensor architecture based on the AI form of GDH where linker between the GDH and AI contains a ligand binding domain that undergoes conformational change following ligand binding. This dislodges the AI from the active site of GDH.

FIG. 4. Utilising a GDH enzyme split site to create an electrochemical biosensor. Reaction conditions were 15 nM GDH-FRB, 10 nM GDH-FKBP with TVMV site

### 1 mM Rapamycin, 1 mM TVMV and 100 mM CaCl₂.

FIG. 5. Electrochemical biosensor comprising a split GDH enzyme, whereby replacement of the engineered, enzymatically inactivating mutant domain (red) by a corresponding active domain (green) enables detection of rapamycin. (A) Binding moieties are FRB and FKBP bind rapamycin. Typically, FRB binds to rapamycin once bound to FKBP. Reaction conditions were 15 nM GDH-FRB, 10 nM GDH-FKBP (pre-cleaved by TVMV), 100 μM CaCl₂ +/−20% serum. (B) Two component system for detection of immunosuppressant drug FK506 (Tacrolimus). The biosensor binding moieties comprise calcineurin A/B heterodimer fused to one active component of GDH and the FKBP fused to another. The right plot represents titration of the sensor with FK506 in the presence or absence of the rapamycin and cyclosporin A. (C) Two component system for detection of immunosuppressant drug cyclosporin A. The sensor is composed of a calcineurin A/B hetero dimer fused to one active component of GDH and the peptidyl-prolyl cis-trans isomerase a fused to another.

FIG. 6. Electrochemical biosensor comprising a split GDH enzyme to detect a amylase. Binding moieties are camelid VHH antibodies designated VHH1 and VHH2 that bind a amylase. Reaction conditions were 20 nM GDH-VHH1 (from PDB: 1KXV), 15 nM GDH-VHH2 (from PDB: 1BVN) (pre-cleaved by TVMV) and 100 uM CaCl₂. (B) Chronoamperometric ananlysis of the amylase biosensor.

Sample preparations and reaction conditions were as follows: Preparation of AMY-2 PQQ/TVMV mix: One aliquot each of AMY-2 PQQ and TVMV (AMY-2 at 20 μL of 50 μM and TVMV at 20 μL of 50 μM) was defrosted. 4 μL of TVMV was mixed with 20 μL of AMY-2 PQQ (final concentrations 8.3 uM TVMV and 41.6 μM AMY-2 PQQ). This was stored at room temperature for 3.00 hours before use. Buffer: 0.01M sodium phosphate buffer (pH 7.4), 142 mM NaCl, 4.2 mM KCl, 2.23 mM MgCl₂.

(A) 25 mM CaCl₂ (MW0034/1, mw 147.01): 50 μM CaCl₂, 2.23 mM MgCl₂ in buffer: 20 uL of 25 mM CaCl₂+9.98 mL buffer. AMY-1: one aliquot (20 μL, 50 μM) was defrosted immediately before use. (B) 500 nM AMY-1: 5 μL of 50uMAMY-1+495 μL of (A). (C) 416 nM of AMY-2 PQQ (83 nM TVMV): 5 μL of AMY-2PQQ/TVMV mix+495 μL of (A). Human salivary alpha amylase (MW0022/1): from Sigma, A1031, lot SLBK8708V. The CofA states the sample is 9% protein and the activity is 852 U/mg. Assuming the molecular weight of alpha amylase is 50 kDa, 0.55 mg/mL of solid contains 1.0 uM alpha amylase. 2.46 uM alpha amylase: 0.00104 g in 0.776 mL (A) (this was prepared immediately before use). 1230 nM alpha amylase: 0.5 mL of 2.46 μM amylase+0.5 mL of (A) 123 nM alpha amylase: 0.1 mL of 1230 nM amylase+0.9 mL of (A).

FIG. 7. Reducing spontaneous GDH enzyme reconstitution by addition of an excess of engineered, enzymatically inactivated mutant domains (red).

FIG. 8. (A) An electrochemical biosensor for detecting a protease target molecule. S=scaffolding domain binding moiety: e.g. SH2 domain, PDZ etc. L=a ligand peptide that binds the scaffolding domain. Cleavage site for protease target molecule is located intermediate S and L so that cleavage allows L coupled to active GDH domain to bind S and facilitate replacement of the engineered, enzymatically inactivating mutant domain (red) by the corresponding active GDH domain (green). (B) activation of thrombin sensor with different concentrations of thrombin (from top to bottom traces 0.013 U/ml; 0.13 U/ml; 1.3 U/ml). In the assay, absorption of 60 μM electron accepting dye dichlorophenolindophenol in the presence of 0.6 mM electron mediator phenazine methosulphate were measured at 600 nm in the presence of 20 mM of glucose, 50 μM CaCl₂, 10 nM GDH-S-L and 15 nM inactive GDH-L. (C) a thrombin sensor carrying a high affinity thrombin binding peptide and a thrombin cleavage site. Activity was measured as in (B) but thrombin concentrations were 0.00013 U/ml; 0.0013 U/ml (0.5 ng/ml; 16 pM); 0.013 U/ml (5 ng/ml); 0.13 U/ml (50 ng/ml); 1.3 U/ml (500 ng/ml). (D) a thrombin sensor carrying two set of high affinity thrombin cleavage site. Activity was measured as in (B) but thrombin concentrations were 0.0013 U/ml (0.5 ng/ml; 10 pM); 0.013 U/ml (5 ng/ml); 0.13 U/ml (50 ng/ml); 1.3 U/ml (500 ng/ml). (E) Activity of factor Xa sensor at different concentrations of the factor Xa. Activity was measured as in (B) but thrombin concentrations were 0.01 μg/ml; 0.1 μg/ml, 1 μg/ml, 7 μg/ml. (F) Same as in E but with a sensor containing three factor Xa cleavage sites. Activity was measured as in (B) but thrombin concentrations were 0.001 μg/ml; 0.01 μg/ml, 0.1 μg/ml, 1 μg/ml, 7 μg/ml.

FIG. 9. Electrochemical biosensor for illicit drug detection. In this embodiment the target molecule is tetrahydrocannabinol (THC). The binding moiety is THC conjugated to a calmodulin binding peptide

FIG. 10. Electrochemical biosensor for illicit drug detection. In this embodiment the target molecule is tetrahydrocannabinol (THC). The binding moiety is a peptide competitively binding to THC antibody.

FIG. 11. Electrochemical biosensor for illicit drug detection. In this embodiment the target molecule is tetrahydrocannabinol (THC). The binding moiety is a peptide competitively binding to an anti-THC antibody and to a scaffolding domain. The third component comprises a THC antibody fused to a protease and a scaffolding domain such as SH2 or PDZ.

FIG. 12. Schematic representation of a basic electronic device for detection of the electric current generated by electrochemical biosensors

FIG. 13. Example of commercial glucose monitor that has been reengineered to contain Cam-GDH biosensor instead of GDH. The sensor was activated by the presence of Ca²⁺ in the human saliva.

FIG. 14. Amino acid sequences of electrochemical biosensors and components thereof (SEQ ID NOS:2-10, 53, 54). GDH amino acid sequences are double underlined, binding moiety amino acid sequences are italicized and TVMV cleavage sites (ETVRFQS; SEQ ID NO:11) are bolded. Amino acid sequences of GDH mutants, GDH fragments, binding moieties, protease cleavage and protease binding sites, and inhibitory peptides (SEQ ID Nos 12-49 and 51-52, 55).

### DETAILED DESCRIPTION

The present invention provides a biosensor which is capable of producing or generating one or more electrons in response to a target molecule. Suitably, the biosensor comprises an enzyme or enzyme fragment switchable between catalytically “inactive” and catalytically “active” states to thereby react with a substrate molecule to produce one or more electrons. Also provided herein are thus enzymes and enzyme fragments having the features of the biosensors described herein. More particularly, the enzyme or enzyme fragment is an oxidoreductase such as glucose dehydrogenase (GDH) which has been engineered to enable switching between catalytically “inactive” and catalytically “active” states. In one particular form the GDH molecule has a heterologous insert which can bind a target molecule, thereby resulting in a conformational change that results in enzyme activation. In another particular form the GDH molecule is a “split enzyme” construct comprising an active portion and an engineered mutant portion, whereby binding of a target molecule by one or more binding moieties of the biosensor results in the engineered mutant portion being replaced by another active portion to thereby reconstitute GDH enzyme activity. The biosensor molecule disclosed herein may have efficacy in molecular diagnostics wherein the “target molecule” is an analyte or other molecule of diagnostic value or importance. However, another application of the biosensor disclosed herein may be in synthetic biology applications for constructing multi-component artificial cellular signalling networks.

It will be appreciated that the indefinite articles “a” and “an” are not to be read as singular indefinite articles or as otherwise excluding more than one or more than a single subject to which the indefinite article refers. For example, “a” molecule includes one molecule, one or more molecules or a plurality of molecules.

As used herein, unless the context requires otherwise, the words “comprise”, “comprises” and “comprising” will be understood to mean the inclusion of a stated integer or group of integers but not the exclusion of any other integer or group of integers.

For the purposes of this invention, by “isolated” is meant material (such as a molecule) that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated proteins and nucleic acids may be in native, chemical synthetic or recombinant form.

By “protein” is meant an amino acid polymer. The amino acids may be natural or non-natural amino acids, D- or L-amino acids as are well understood in the art.

A “peptide” is a protein having less than fifty (50) amino acids.

A “polypeptide” is a protein having fifty (50) or more amino acids.

An “enzyme” is a protein having catalytic activity towards one or more substrate molecules. Suitably, the enzyme is capable of displaying catalytic activity towards a substrate molecule to thereby produce one or more electrons. In some embodiments, the enzyme is an oxidoreductase. In one particular embodiment the enzyme is glucose dehydrogenase (GDH) and the substrate molecule is glucose. The catalytic activity may thus be glucose dehydrogenase activity which may be measured in accordance with Example 1. The glucose dehydrogenase may be a PQQ-GDH or an FAD-GDH. Preferably, the GDH is a PQQ-GDH. In another embodiment the enzyme is glucose oxidase and the substrate is glucose. In another embodiment the enzyme is dihydrofolate reductase (DHFR) and the substrate molecule is dihydrofolic acid. In another embodiment the enzyme is lactate dehydrogenase (LDH) and the substrate molecule is lactate.

As generally used herein “catalytically active” and “catalytically active state” may refer to absolute or relative amounts of enzyme activity that can be displayed or achieved by an enzyme or a fragment or portion thereof. Typically, an enzyme is catalytically active or in a catalytically active state if it is capable of displaying specific enzyme activity towards a substrate molecule to produce one or more electrons under appropriate reaction conditions. As generally used herein “catalytically inactive” and “catalytically inactive state” may refer to an enzyme, fragment or portion thereof that is substantially incapable of displaying specific enzyme activity towards a substrate molecule under appropriate reaction conditions. Typically, the electrons produced would be substantially less compared to that produced by a corresponding catalytically active enzyme, or would be entirely absent.

A first aspect, the invention provides a biosensor molecule comprising at least one amino acid sequence of an enzyme capable of reacting with a substrate molecule when in a catalytically active state to produce one or more electrons; and at least one heterologous, modifier amino acid sequence that releasably maintains the enzyme in a catalytically inactive state, wherein the heterologous, modifier amino acid sequence is responsive to a target molecule to switch the amino acid sequence of the enzyme from the catalytically inactive state to said catalytically active state.

Suitably, the at least one enzyme amino acid sequence and said at least one heterologous, sensor amino acid sequence are present in, or form at least part of a single, contiguous amino acid sequence.

In a particular embodiment, said at least one heterologous, sensor amino acid sequence is an insert in said at least one enzyme amino acid sequence, to thereby facilitate switching the enzyme amino acid sequence between said catalytically inactive and said catalytically active state. In this context, by “insert” is meant an amino acid sequence that is heterologous to said at least one enzyme amino acid sequence is located between, and contiguous with, respective portions, sub-sequences or fragments of said at least one enzyme amino acid sequence.

Suitably, the heterologous, sensor amino acid sequence binds said target molecule to thereby switch the amino acid sequence of the enzyme from the catalytically inactive state to said catalytically active state. Preferably, the binding of the target molecule by the heterologous, sensor amino acid sequence results in a conformational change which results in, or facilitates, switching the amino acid sequence of the enzyme from the catalytically inactive state to said catalytically active state.

In one embodiment, the heterologous, sensor amino acid sequence is an amino acid sequence of a calcium-binding protein, or a fragment thereof. According to this embodiment, the target molecule is, or comprises, calcium. In a particular embodiment, the calcium-binding protein is calmodulin.

The enzyme may be any enzyme capable of reacting with a substrate molecule to thereby produce one or more electrons. Preferably, the enzyme is an oxidoreductase such as a GDH, LDH or DHFR. According to these embodiments, the substrate molecule is respectively glucose, lactate or dihydrofolic acid. Where the enzyme is a GDH, it may be an FAD-GDH or a PQQ-GDH. A PQQ-GDH preferably comprises the sequence of SEQ ID NO: 1 or a variant thereof.

The invention accordingly further provides an oxidoreductase enzyme, preferably a glucose dehydrogenase (GDH) enzyme comprising a heterologous, sensor amino acid sequence which is responsive to a target molecule, wherein binding of the target molecule acts to regulate catalytic activity of the enzyme.

The target molecule may be any target molecule described herein, and accordingly the heterologous sensor amino acid sequence responsive to said target molecule may be any binding moiety for said target molecule described herein, which when comprised in the enzyme has the ability to releasably regulate catalytic activity of the enzyme dependent on interaction with the target molecule. The heterologous, sensor amino acid sequence thus reversibly regulates catalytic activity of the enzyme. The heterologous, sensor amino acid sequence can be displaced in the presence of the target molecule to thereby catalytically activate the enzyme. The heterologous, sensor amino acid sequence can allosterically regulate the catalytic activity of the enzyme. The heterologous, sensor amino acid sequence may comprise one more domains (such as one or two domains) which undergo structural rearrangement upon binding of a target molecule such as a peptide or protein. Alternatively, the heterologous, sensor amino acid sequence may represent an unstructured or unfolded sequence which undergoes a structural rearrangement upon binding of a target molecule such as a peptide or protein. The structural rearrangement may create one or more folded protein domains.

The heterologous, sensor amino acid sequence may be a binding moiety as described below. The heterologous, sensor amino acid sequence may be an affinity clamp as described below. The heterologous, sensor amino acid sequence is preferably an amino acid sequence of a calcium-binding protein, or a functional fragment thereof. The calcium binding protein may be a calmodulin or a functional calcium-binding fragment thereof.

The heterologous, sensor amino acid sequence is typically provided as an insert within the amino acid sequence of the enzyme. The insertion is made at a position in the amino acid sequence of the enzyme which tolerates said insertion without steric clashes preventing stable folding of the enzyme. Linker sequences may be added between the insert and the sequence of the enzyme to assist toleration of the insertion. The insertion typically allows for the heterologous, sensor amino acid sequence to reversibly inhibit catalytic activity through inducing a conformational change in the enzyme, typically at the active site of the enzyme. The heterologous, sensor amino acid sequence typically undergoes a conformational change in the presence of the target molecule which releases its inhibitory effect on the enzyme and restores catalytic activity. The insertion may be located at a loop or turn region in the structure of the enzyme which functionally tolerates the heterologous, sensor amino acid sequence, as described above.

In one particular embodiment of the biosensor and enzyme described above, a glucose dehydrogenase, for example a pyrroloquinoline quinone glucose dehydrogenase (PQQ-GDH) such as Acinetobacter calcoaceticus PQQ-GDH may be engineered with an allosteric receptor domain to control catalytic activity in response to target molecule binding. The present inventors have analyzed a high resolution structure of A. calcoaceticus PQQ-GDH (PDB: 1CQ1) for possible sites in the vicinity of the active center of the enzyme that would be close enough to transmit conformational changes to the active center while tolerating insertion of a heterologous, sensor amino acid sequence. The loop connecting strands A and B of the β-sheet 3 is proposed as a suitable site for this insertion. The beginning of strand A harbors His144 that acts as a general base that extracts a proton from the glucose O1 atom. As His144 is critical for catalysis, its dislocation via torsion introduced by separation of strands A and B leads to a change of GDH catalytic activity.

In one embodiment, the biosensor comprises a calcium-binding domain of calmodulin inserted into the loop connecting strands 3A and 3B, so that binding of calcium by this domain causes a substantial conformational change. In a particular embodiment, the biosensor is a chimeric protein where residues 12-67 of mouse CaM are inserted between residues 153 and 155 of PQQ-GDH. In order to reduce structural tension and clashes, we also introduced a GSGS linker at N-terminal of calmodulin and a Gly linker at the C-terminus of the calmodulin amino acid sequence in the junction site. In the absence of Ca²⁺ ions the biosensor displays virtually no enzymatic activity. Addition of Ca²⁺ ions results in dose-dependent activation of the biosensor while having only limited effect on PQQ-GDH lacking the heterologous calmodulin insert.

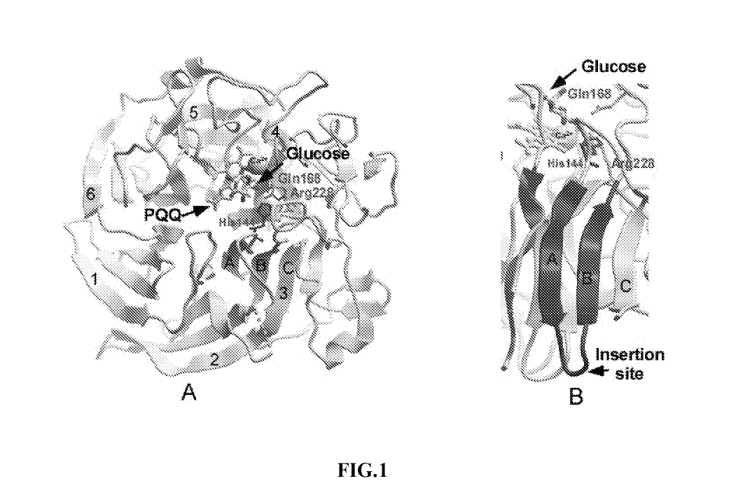

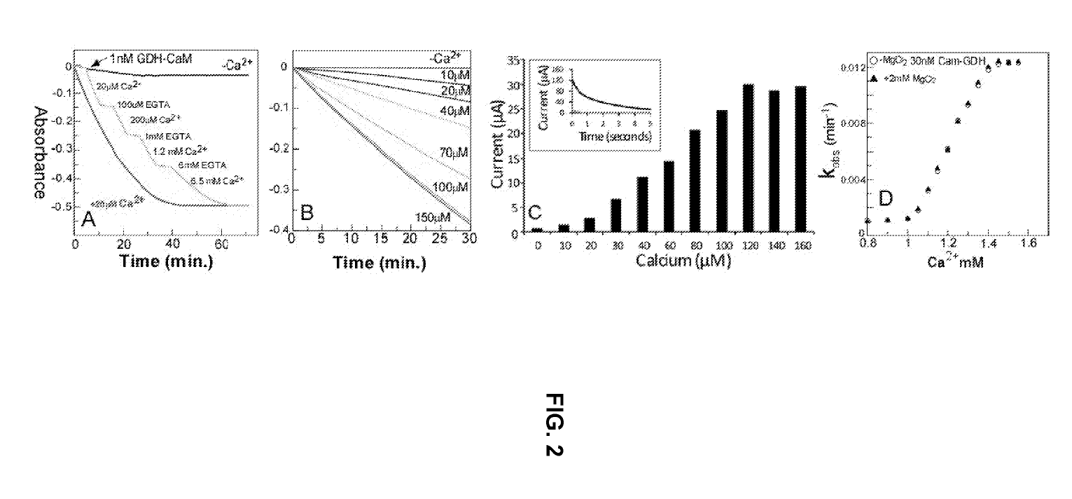

Non-limiting examples of these embodiments are shown in FIGS. 1-3. Any heterologous, sensor amino acid sequence described herein may be introduced at a loop or turn region of a GDH enzyme corresponding to the loop connecting strands 3A and 3B of PQQ-GDH as described above or in a region corresponding to residues 153 to 155 of said enzyme (residues 153-155 of SEQ ID NO:1). The skilled person is able to identify corresponding locations in other enzymes from structural analysis and sequence alignment. A corresponding location is typically one which accommodates the inserted heterologous, sensor amino acid sequence such that it reversibly inhibits catalytic activity of the enzyme as described above. The insertion may dislocate a catalytic residue corresponding to His144 of SEQ ID NO:1.

The invention further provides a GDH enzyme comprising a heterologous sensor amino acid sequence inserted in between residues 153 and 155 of SEQ ID NO:1 or a variant thereof. Such an enzyme may comprise in order the sequences of SEQ ID NO: 13 (residues 1-153) or a variant thereof, the heterologous sensor amino acid sequence, and SEQ ID NO: 15 (residues 155-454) or a variant thereof. Variants of SEQ ID Nos 1, 13 and 15 are further described below. The above sequences may be separated by linker sequences allowing for toleration of the inserted heterologous sensor amino acid sequence as described above. The invention further provides a calcium biosensor based on PQQ-GDH and mouse CaM as described above comprising SEQ ID NO: 53 or a variant thereof.

The invention additionally provides a method of engineering an oxidoreductase enzyme, preferably a glucose dehydrogenase (GDH) enzyme comprising a heterologous, sensor amino acid sequence which is responsive to a target molecule, wherein binding of the target molecule acts to regulate catalytic activity of the enzyme. The method comprises selecting a suitable location in the enzyme able to tolerate insertion of the heterologous, sensor amino acid sequence, and inserting said heterologous, sensor amino acid sequence into the enzyme, such that an enzyme is engineered which responds to the target molecule to regulate (typically activate) catalytic activity of the enzyme.

The invention further provides a biosensor molecule comprising at least one amino acid sequence of an enzyme capable of reacting with a substrate molecule when in a catalytically active state; and at least one other amino acid sequence of said enzyme which is engineered to releasably maintain the enzyme in a catalytically inactive state, wherein the biosensor is responsive to a target molecule to switch the amino acid sequence of the enzyme from the catalytically inactive state to said catalytically active state. The at least one amino acid sequence and at least one other (engineered) amino acid sequence of said enzyme may non-covalently interact, and the engineered amino acid sequence be replaced by a yet another amino acid sequence as further described below in the context of electrochemical biosensors.

A preferred aspect of the invention provides a biosensor molecule comprising at least one amino acid sequence of an enzyme capable of reacting with a substrate molecule when in a catalytically active state to produce one or more electrons; and at least one other amino acid sequence of said enzyme which is engineered to releasably maintain the enzyme in a catalytically inactive state, wherein the biosensor is responsive to a target molecule to switch the amino acid sequence of the enzyme from the catalytically inactive state to said catalytically active state.

Suitably, said at least one other amino acid sequence of said enzyme is engineered to comprise one or more amino acid sequence mutations. Suitably, said at least one amino acid sequence of the enzyme capable of reacting with a substrate molecule when in a catalytically active state to produce one or more electrons and said at least one other amino acid sequence of said enzyme engineered to releasably maintain the enzyme in a catalytically inactive state, non-covalently interact.

In an embodiment, these are respective amino acid sequences of the enzyme, one of which is engineered to comprise one or more amino acid sequence mutations to thereby inhibit, prevent or otherwise suppress said least one amino acid sequence of the enzyme reacting with the substrate molecule. Suitably, this “engineered mutant” non-covalently associates with said at least one amino acid sequence of the enzyme capable of reacting with the substrate molecule, thereby acting to inhibit, prevent or otherwise suppress enzyme activity. In a preferred embodiment, the respective amino acid sequences of the enzyme are expressed or otherwise produced as a single, contiguous amino acid sequence that is subsequently cleaved by a protease to thereby enable the non-covalent association between the engineered mutant and said at least one amino acid sequence of the enzyme capable of reacting with the substrate molecule.

The said at least one amino acid sequence of the enzyme thus typically represents a first fragment sequence of said enzyme which is able to non-covalently interact with a said at least one other or a said yet another amino acid sequence of said enzyme representing a second fragment sequence of said enzyme, to reconstitute a stable enzyme. The first and second fragment sequences may together constitute the complete sequence of the enzyme or together constitute sufficient sequence of the enzyme to provide for a stable form of said enzyme including its catalytic domain.

The reconstituted enzyme may be a stable non-catalytically active enzyme where the first fragment sequence represents a said at least one other amino acid sequence of said enzyme engineered to releasably maintain the enzyme in a catalytically inactive state. Alternatively, the reconstituted enzyme may be a stable catalytically active enzyme where the first fragment sequence represents said yet another amino acid sequence of said enzyme capable of replacing the said at least one other amino acid sequence of said enzyme engineered to releasably maintain the enzyme in a catalytically inactive state. A stable enzyme as described herein is one in which said non-covalently interacting amino acid sequences form a soluble enzyme complex. The non-covalently interacting amino acid sequences may further be reversibly dissociated by another, replacing, amino acid sequence to form an alternative stable enzyme.

In an embodiment relating to GDH, the respective amino acid sequences of the enzyme may be the sequences of SEQ ID NO: 13 or a variant thereof, and SEQ ID NO:15 or a variant thereof. The “engineered mutant” typically comprises a H144 mutation and a mutation to one or more of Q76 and D143. The mutations are selected to reduce or abolish catalytic activity of the enzyme. Preferably, H144, Q76 and D143 are each mutated. These residues may be each mutated to alanine, or alternative mutations to alanine which reduce or abolish catalytic activity can be made. The engineered mutant may comprise the sequence of SEQ ID NO: 14 or a variant thereof which also produces a catalytically inactive enzyme when non-covalently associated with the at least one amino acid sequence of the enzyme. The variant may comprise alternative mutations to those in SEQ ID NO: 14 at positions 76, 143 and 144, as described herein.

The resulting, engineered mutant is preferably expressed in bacteria such as E. coli as en epitope-tagged protein and is purified by affinity chromatography.

In an embodiment, the protease cleavage site is a TVMV cleavage site such as ETVRFQS (SEQ ID NO:11) or a functional variant thereof. The protease cleavage site may alternatively be a Thrombin cleavage site such as SEQ ID NO: 33 or a functional variant thereof, or Factor Xa site such as SEQ ID NO: 34 or 35 or a functional variant thereof.

In one embodiment, the biosensor of this aspect further comprises yet another amino acid sequence of said enzyme which is capable of replacing said at least one other amino acid sequence of said enzyme engineered to releasably maintain the enzyme in a catalytically inactive state. Typically, said yet another amino acid sequence of said enzyme comprises an amino acid sequence that substantially corresponds to that of the engineered mutant, although lacking the one or more amino acid sequence mutations. Typically, this replacement restores the catalytic activity of the enzyme by non-covalently combining said yet another amino acid sequence of said enzyme with said at least one amino acid sequence of the enzyme capable of reacting with a substrate to form a functional, catalytically active enzyme.

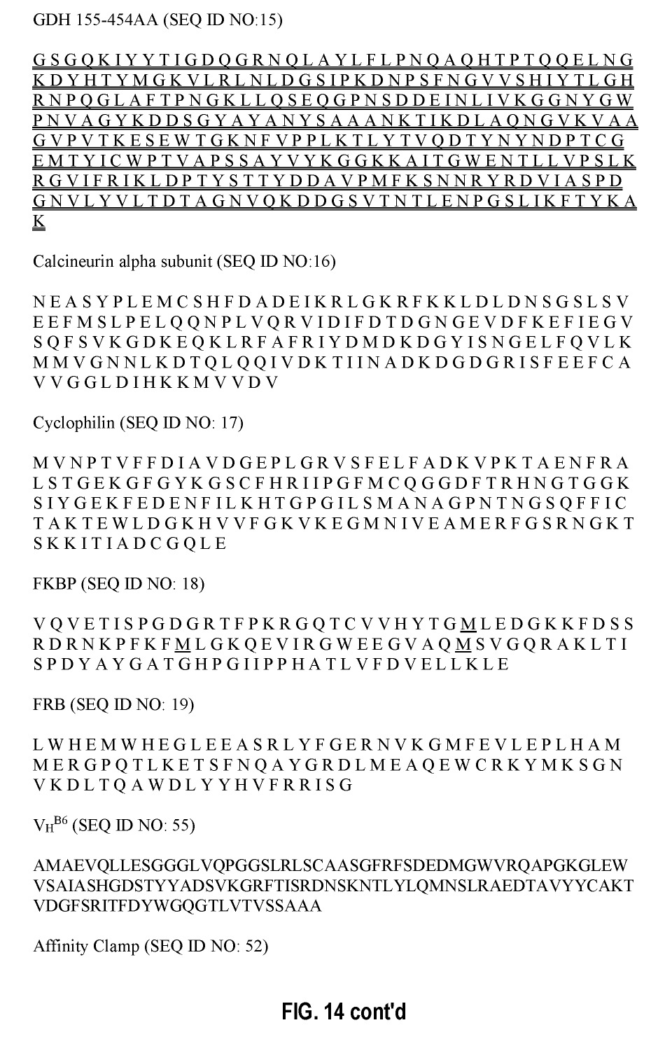

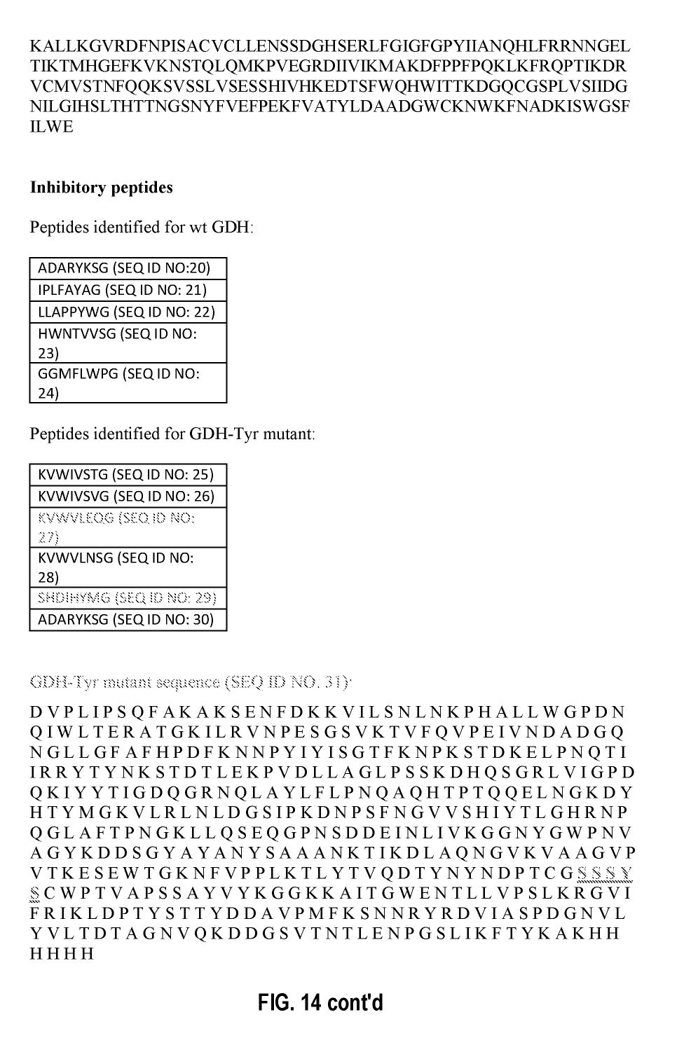

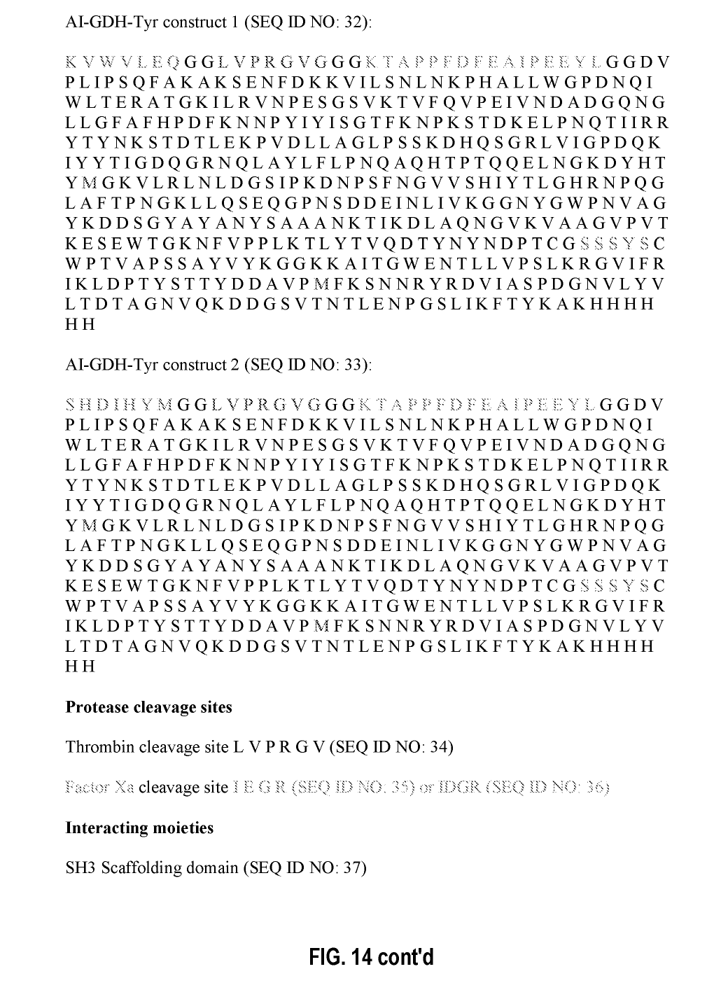

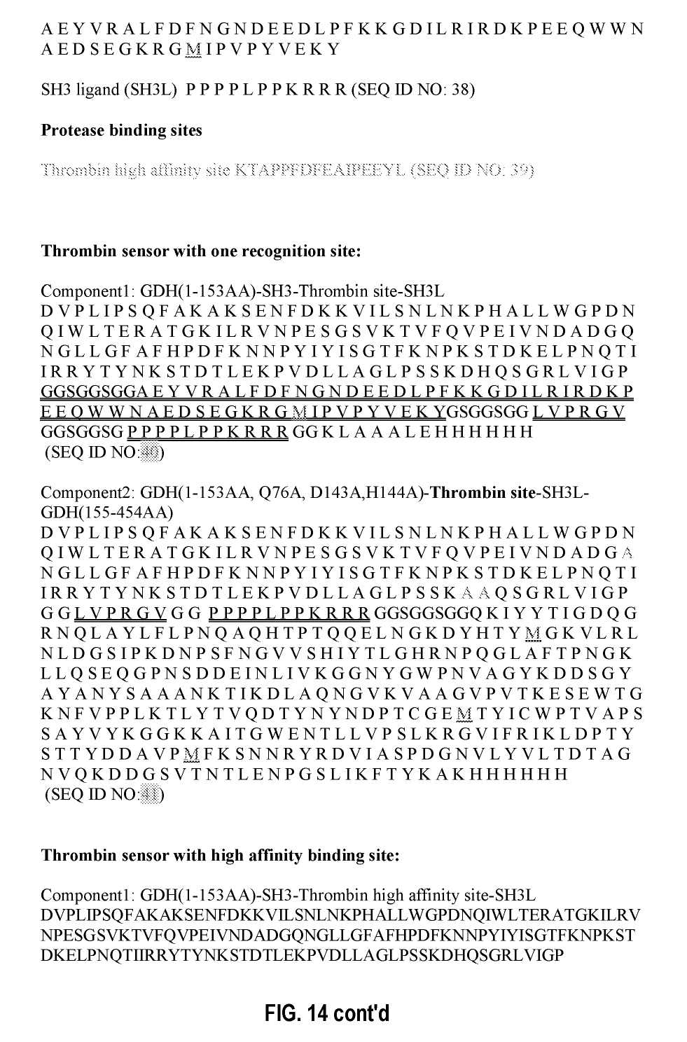

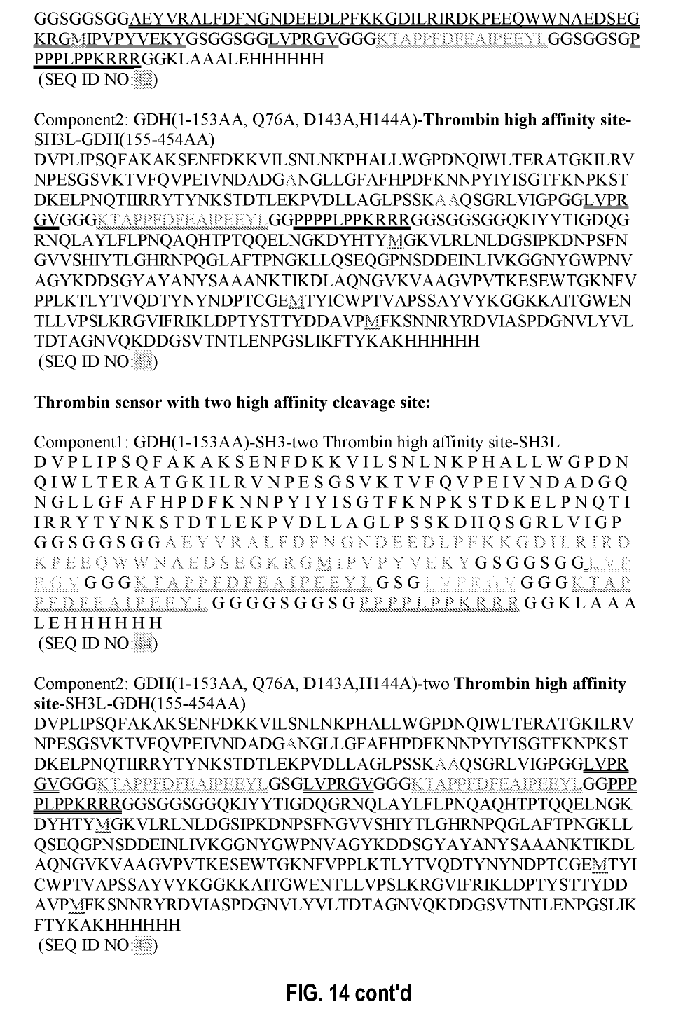

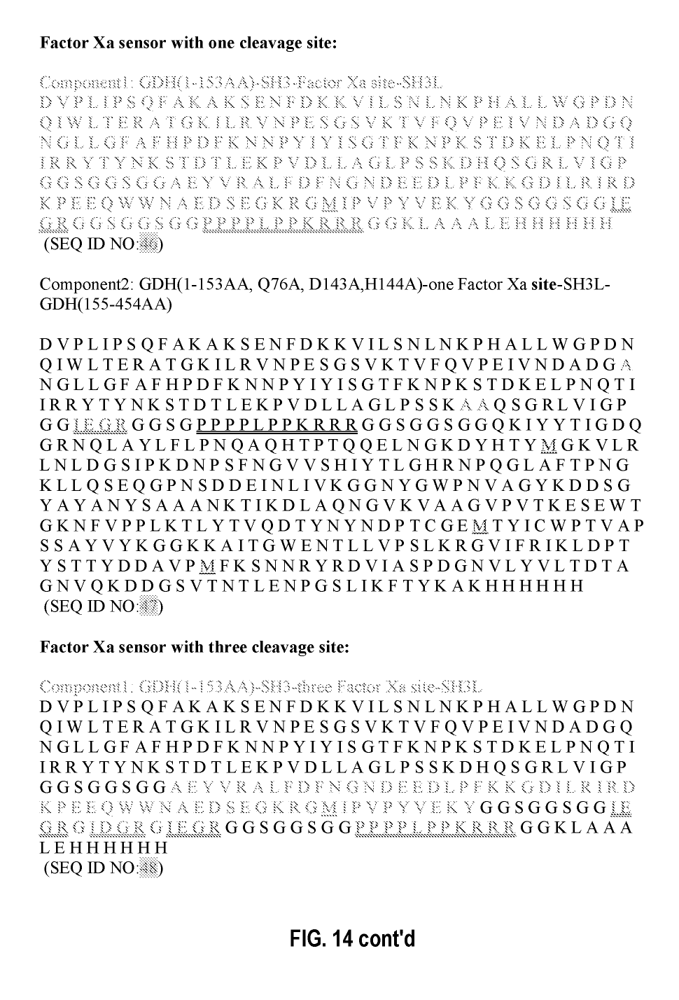

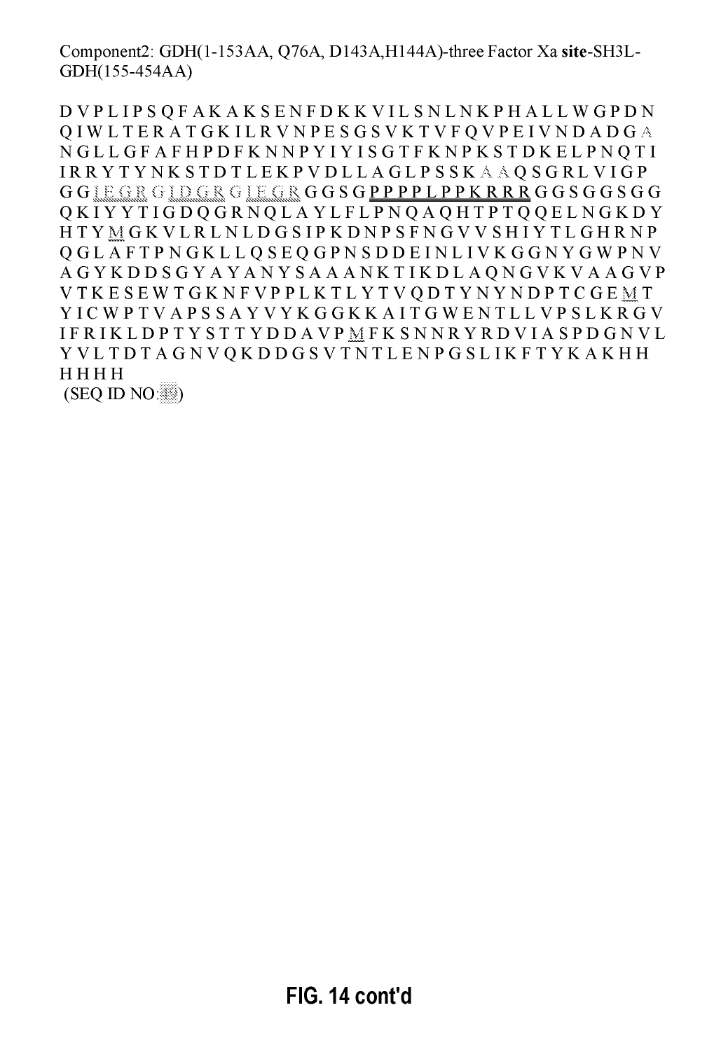

Non-limiting examples of such embodiments are shown in FIGS. 4-9. With particular regard to FIG. 7, it will be appreciated that a molar excess of engineered mutants may be provided that reduce or eliminate spontaneous replacement of the catalytic activity of the enzyme in the absence of target molecule binding. This thereby suppresses “background noise”, thus improving the sensitivity of the biosensor.

In a broad embodiment, said yet another amino acid sequence of said enzyme and said at least one amino acid sequence of the enzyme capable of reacting with a substrate comprise respective binding moieties that can interact, such as by binding a target molecule, to facilitate the replacement of the engineered amino acid sequence by said yet another amino acid sequence.

In a related aspect, the invention further provides a polypeptide comprising a first fragment sequence of an oxidoreductase enzyme, preferably a glucose dehydrogenase (GDH) enzyme, which is capable of non-covalently interacting with a polypeptide comprising a second fragment sequence of said enzyme to reconstitute a stable enzyme. The first and second fragment sequences may together constitute the complete sequence of the enzyme or together constitute sufficient sequence of the enzyme to provide for a stable form of said enzyme including its catalytic domain, as described above.

The polypeptide comprising a first fragment sequence may be capable of reconstituting a stable catalytically active enzyme with said polypeptide comprising a second fragment sequence of said enzyme. In this embodiment, the polypeptide comprising a first fragment sequence of said enzyme is able to displace a corresponding fragment sequence of said enzyme which is engineered to maintain an enzyme in a catalytically inactive state from a stable enzyme complex, to restore catalytic activity. The polypeptide comprising a first fragment sequence may alternatively comprise one or more mutations as defined above which render a stable enzyme comprising said polypeptide catalytically inactive. Such a polypeptide (also described as an engineered polypeptide)_is also able to be displaced from said stable enzyme complex to restore catalytic activity.

Also provided is an oxidoreductase enzyme, preferably a GDH enzyme which comprises both a first fragment sequence which is engineered as described above, and also a said second fragment sequence as part of a contiguous polypeptide, where the first and second fragment sequences are separated by one or more protease cleavage sites, such that protease activity allows for the engineered fragment sequence to be displaced, and a first fragment sequence capable of restoring catalytic activity to then non-covalently associate with the second fragment sequence to form a stable catalytically active enzyme.

The polypeptides described above may comprise a binding moiety capable of interacting with a respective binding moiety on a counterpart polypeptide comprising a second fragment sequence of said enzyme, wherein the interaction between the binding moieties regulates catalytic activity of the reconstituted stable glucose dehydrogenase enzyme. The interaction between the binding moieties may be regulated by binding of a target molecule. The binding moieties and corresponding target molecule may be selected from any described herein.

A polypeptide described above may further comprises a sequence inhibiting interaction of the respective binding moieties, and one or more protease cleavage sites, wherein cleavage by the protease provides for interaction between the binding moieties. The polypeptide may further comprise a sequence enhancing binding and/or cleavage efficiency of the protease. The protease cleavage site and the sequence enhancing binding and/or cleavage efficiency of the protease may be selected from any described herein.

In particular embodiments, the first and second fragment sequences described above may be derived by cleavage of a GDH enzyme in a loop or turn region of a GDH enzyme corresponding to the loop connecting strands 3A and 3B of PQQ-GDH as described above or in a region corresponding to residues 153 to 155 of said enzyme (residues 153-155 of SEQ ID NO:1). The skilled person is able to identify corresponding locations in other enzymes from structural analysis and sequence alignment. A corresponding location is typically one which allows for generation of functional fragments of said enzyme which are able to reconstitute a stable enzyme.

In this aspect, the invention additionally provides a method of engineering an oxidoreductase enzyme, preferably a glucose dehydrogenase (GDH) enzyme to provide first and second fragment sequences capable of reconstitute a stable enzyme. The method comprises selecting a suitable location in the enzyme at which the enzyme may be cleaved to provide said first and second fragment sequences. The method typically further comprises introducing mutations into one of said sequences which render a stable enzyme reconstituted from said sequence catalytically inactive. The method may further comprise adding one or more binding moieties to said sequences which assist non-covalent association of polypeptides comprising the sequences to reconstitute a stable catalytically active enzyme.

The invention further provides a polypeptide comprising a first fragment sequence of a GDH enzyme which comprises SEQ ID NO: 13 or a variant thereof. This polypeptide may be a polypeptide capable of reconstituting a stable catalytically active GDH enzyme as described above. The invention additionally provides a polypeptide comprising a first fragment sequence of a GDH enzyme which comprises SEQ ID NO: 14 or a variant thereof. This polypeptide may be engineered to render a stable enzyme comprising said polypeptide catalytically inactive as described above. A variant of SEQ ID NO: 14 may comprise alternative inactivating mutations to alanine at one or more of, preferably all of H144, Q76 and D143 as described above. A variant of SEQ ID NO: 13 or 14 may be a sequence which when included in a said polypeptide is capable of reconstituting a stable GDH enzyme together with a polypeptide comprising SEQ ID NO: 15.

The invention further provides a polypeptide comprising a second fragment sequence of a GDH enzyme which comprises SEQ ID NO: 15 or a variant thereof. A variant of SEQ ID NO: 15 may be a sequence which when included in a said polypeptide is capable of reconstituting a stable GDH enzyme together with a polypeptide comprising SEQ ID NO: 13 or SEQ ID NO: 14 as described above.

The above polypeptides comprising SEQ ID NO: 13 or a variant thereof, SEQ ID NO: 14 or a variant thereof, or SEQ ID NO: 15 or a variant thereof may further comprise one or more binding moieties selected from any described herein. Typically, a binding moiety is provided C-terminal to the sequence of SEQ ID NO: 13 or SEQ ID NO: 14 or variant thereof, and N-terminal to the sequence of SEQ ID NO: 15 or variant thereof in a said polypeptide. Representative examples of such polypeptides including binding moieties are provided by SEQ ID NOs 2, 4, 7, 9. The invention further encompasses variants of any of SEQ ID NOs 2, 4, 7, and 9 as described herein.

A polypeptide comprising SEQ ID NO: 13 or a variant thereof is also provided which further comprises two cognate (respective) binding moieties separated by one or more, such as one, two or three protease cleavage sites. The polypeptide may additionally comprise a sequence enhancing binding and/or cleavage efficiency of the protease. The cognate binding moieties interact in the absence of the protease, which interaction is then disrupted by cleavage of the protease to allow for binding of a retained binding moiety to a respective binding moiety on a further polypeptide comprising SEQ ID NO: 15 or a variant thereof, to thereby reconstitute a catalytically active GDH enzyme. The cognate binding moieties, protease cleavage sites and sequences enhancing binding and/or cleavage efficiency may be selected from any described herein. Representative examples of the above polypeptides are provided by SEQ ID NOs 40, 42, 44, 46, and 48. The invention further encompasses variants of any of SEQ ID NOs 40, 42, 44, 46, and 48 as described herein.

Also provided is a GDH enzyme comprising the sequence of SEQ NO: 14 or a variant thereof, and additionally the sequence of SEQ ID NO: 15 or a variant thereof, wherein one or more protease cleavage sites are located between said sequences, such that cleavage by a protease is able to displace a polypeptide comprising the sequence of SEQ ID NO: 14 from said enzyme. The GDH enzyme may further comprise a binding moiety capable of interacting with a respective binding moiety on a polypeptide comprising a first fragment sequence of a GDH enzyme which comprises SEQ ID NO: 13 or a variant thereof, optionally in the presence of a target molecule, wherein interaction between the binding moieties allows for reconstitution of a stable GDH enzyme. Representative examples of such GDH enzymes are provided by SEQ ID NOs 3, 6, 9, 41, 43, 45, 47 and 49. The invention further encompasses variants of any of SEQ ID NOs 3, 6, 9, 41, 43, 45, 47 and 49 as described herein.

The above polypeptides and enzymes may be provided on a biosensor as described herein. Alternatively, suitable combinations of polypeptides and enzymes which interact together to detect a target molecule as described herein and in the representative example biosensors may be provided together in any in vitro context, in which detection of the target molecule is possible. The polypeptides and enzymes may be provided together in solution for detection of a target molecule. As generally used herein a “binding moiety” or “binding moieties” refer to one or a plurality of molecules or biological or chemical components or entities that are capable of recognizing and/or binding each other, or one or more other target molecules. Binding moieties may be proteins, nucleic acids (e.g single-stranded or double-stranded DNA or RNA), sugars, oligosaccharides, polysaccharides or other carbohydrates, lipids or any combinations of these such as glycoproteins, PNA constructs etc or molecular components thereof By way of example only, binding moieties may be, or comprise: (i) an amino acid sequence of a ligand binding domain of a receptor responsive to binding of a target molecule such as a cognate growth factor, cytokine, a hormone (e.g. insulin), neurotransmitters etc; (ii) an amino acid sequence of an ion or metabolite transporter capable of, or responsive to, binding of a target molecule such as an ion or metabolite (e.g a Ca²⁺-binding protein such as calmodulin or calcineurin or a glucose transporter); (iii) a zinc finger amino acid sequence responsive to zinc-dependent binding a DNA target molecule; (iv) a helix-loop-helix amino acid sequence responsive to binding a DNA target molecule; (v) a pleckstrin homology domain amino acid sequence responsive to binding of a phosphoinositide target molecule; (vi) an amino acid sequence of a Src homology 2- or Src homology 3-domain responsive to a signaling protein; (vii) an amino acid sequence of an antigen responsive to binding of an antibody target molecule; or (viii) an amino acid sequence of a protein kinase or phosphatase responsive to binding of a phosphorylatable or phosphorylated target molecule; (ix) ubiquitin-binding domains; (x) proteins or protein domains that bind small molecules, drugs or antibiotics such as rapamycin-binding FKBP and FRB domains; (xi) single- or double-stranded DNA, RNA or PNA constructs that bind nucleic acid target molecules, such as where the DNA or RNA are coupled or cross-linked to an amino acid sequence or other protein-nucleic acid interaction; and/or (xii) an affinity clamp such as a PDZ-FH3 domain fusion; inclusive of modified or engineered versions thereof, although without limitation thereto.

Particular binding moieties of use in the invention are provided by SEQ ID NOs 5, 16-19, 36-37, 52 and 55 and variants thereof. Variants are typically functionally binding variants for the relevant respective binding moiety.

It will also be appreciated that binding moieties may be modified or chemically derivatized such as with binding agents such as biotin, avidin, epitope tags, lectins, carbohydrates, lipids although without limitation thereto.

In some embodiments, respective moieties may directly bind, interact or form a complex. The first binding moiety and the second binding moiety may comprise molecules that can directly bind or interact. Accordingly, the direct binding interaction between the target molecule and the binding moieties suitably facilitates co-localization of the first and second molecular components.

In other embodiments, the respective binding moieties are capable of binding, interacting or forming a complex with a target molecule. Typically, the respective binding moieties are capable of binding, interacting or forming a complex with the same target molecule. It will also be appreciated that the “same” target molecule can have respective, different moieties, subunits, domains, ligands or epitopes that can be bound by the respective binding moieties to thereby co-localize and activate protease activity.

The target molecule may be any ligand, analyte, small organic molecule, epitope, domain, fragment, subunit, moiety or combination thereof, such as a protein inclusive of antibodies and antibody fragments, antigens, enzymes, phosphoproteins, glycoproteins, lipoproteins and glycoproteins, lipid, phospholipids, carbohydrates inclusive of simple sugars, disaccharides and polysaccharides, nucleic acids, nucleoprotein or any other molecule or analyte. These include drugs and other pharmaceuticals including antibiotics, banned substances, illicit drugs or drugs of addiction, chemotherapeutic agents and lead compounds in drug design and screening, molecules and analytes typically found in biological samples such as biomarkers, tumour and other antigens, receptors, DNA-binding proteins inclusive of transcription factors, hormones, neurotransmitters, growth factors, cytokines, receptors, metabolic enzymes, signaling molecules, nucleic acids such as DNA and RNA, membrane lipids and other cellular components, pathogen-derived molecules inclusive of viral, bacterial, protozoan, fungal and worm proteins, lipids, carbohydrates and nucleic acids, although without limitation thereto. As previously, described, it will be appreciated that the “same” target molecule can be bound by different, respective binding moieties.

In one embodiment, the binding moieties comprise an amino acid sequence of at least a fragment of any protein or protein fragment or domain that can bind or interact directly, or bind to a target molecule. The binding moiety may be, or comprise a protein such as a peptide, antibody, antibody fragment or any other protein scaffold that can be suitably engineered to create or comprise a binding portion, domain or region (e.g. reviewed in Binz et al., 2005 Nature Biotechnology, 23, 1257-68) which binds a target molecule.

In one particular embodiment, the binding moieties respectively are, or comprise, amino acid sequences of an affinity clamp. The affinity clamp preferably comprises a recognition domain and, optionally, an enhancer domain. The recognition domain is typically capable of binding one or more target molecules, such as described in (i)-(ix) above. Recognition domains may include, but are not limited to, domains involved in phospho-tyrosine binding (e.g. SH2, PTB), phospho-serine binding (e.g. UIM, GAT, CUE, BTB/POZ, VHS, UBA, RING, HECT, WW, 14-3-3, Polo-box), phospho-threonine binding (e.g. FHA, WW, Polo-box), proline-rich region binding (e.g. EVH1, SH3, GYF), acetylated lysine binding (e.g. Bromo), methylated lysine binding (e.g. Chromo, PHD), apoptosis (e.g. BIR, TRAF, DED, Death, CARD, BH), cytoskeleton modulation (e.g. ADF, GEL, DH, CH, FH2), ubiquitin-binding domains or modified or engineered versions thereof, or other cellular functions (e.g. EH, CC, VHL, TUDOR, PUF Repeat, PAS, MH1, LRR1, IQ, HEAT, GRIP, TUBBY, SNARE, TPR, TIR, START, SOCS Box, SAM, RGS, PDZ, PB1, LIM, F-BOX, ENTH, EF-Hand, SHADOW, ARM, ANK).

The enhancer domain typically increases or enhances the binding affinity for at least one or the target molecules. In some embodiments, the affinity may be increased by at least 10, 100 or 1000 fold compared to that of the recognition domain alone. The affinity clamp may further comprise linker connecting the recognition domain and the enhancer domain.

In one particular embodiment, the affinity clamp comprises a recognition domain that comprises at least a portion or fragment of a PDZ domain and an enhancer domain that comprises at least a portion or fragment of a fibronectin type III domain. The PDZ domain may be derived from a human Erbin protein. Erbin-PDZ (ePDZ) binds to target molecules such as the C-termini of p120-related catenins (such as δ-catenin and Armadillo repeat gene deleted in Velo-cardio-facial syndrome (ARVCF)). Preferably, this embodiment of the affinity claim further comprises the tenth (10^{th}) type III (FN3) domain of human fibronectin as an enhancer domain.

In some embodiments, the affinity clamp may comprise one or more connector amino acid sequences. For example, a connector amino acid sequence may connect the protease amino acid sequence (such as comprising a protease amino acid sequence) to the Erbin-PDZ domain, the Erbin-PDZ domain to the FN3 domain and/or the FN3 domain to the inhibitor.

Reference is also made to WO2009/062170, Zhuang & Liu, 2011, Comput. Theoret. Chem. 963 448, Huang et al, 2009, J. Mol. Biol. 392 1221, Huang et al., 2008, PNAS (USA) 105 6578, and Koidel, * and Huang Methods Enzymol. 2013; 523: 285-302 for a more detailed explanation of affinity clamp structure and function, and of particular affinity clamps that may be used in accordance with the invention. An example of an affinity clamp that may be employed in the invention is provided as SEQ ID NO: 52.

In another embodiment, the binding moieties comprise one or a plurality epitopes that can be bind or be bound by an antibody target molecule.

In another embodiment, the binding moieties may be or comprise an antibody or antibody fragment, inclusive of monoclonal and polyclonal antibodies, recombinant antibodies, Fab and Fab′2 fragments, diabodies and single chain antibody fragments (e.g. scVs), although without limitation thereto. Suitably, the first and second binding moieties may be or comprise respective antibodies or antibody fragments that bind a target molecule. Non-limiting examples are shown schematically in FIG. 2C.

In yet another particular embodiment, the binding moieties may be or comprise an antibody-binding molecule, wherein the antibody(ies) has specificity for a target molecule. The antibody-binding molecule is preferably an amino acid sequence of protein A, or a fragment thereof (e.g a ZZ domain), which binds an Fc portion of the antibody.

A particular embodiment of this aspect therefore provides a biosensor comprising a first component that comprises: at least one amino acid sequence of an enzyme capable of reacting with a substrate molecule when in a catalytically active state to produce one or more electrons; a first binding moiety; and at least one other amino acid sequence of said enzyme which is engineered to releasably maintain the enzyme in a catalytically inactive state; and a second component comprising at least one other amino acid sequence of said enzyme and a second binding moiety; arranged so that an interaction between said first and second binding moieties facilitates replacement of said at least one other amino acid sequence of the first component by said at least one other amino acid sequence of the second component, to thereby switch the enzyme of the first component from a catalytically inactive state to a catalytically active state.

While the terms “first”, “second” and “third” are used in the context of respective, separate or discrete molecular components and/or binding moieties of the biosensor, it will be appreciated that these do not relate to any particular non-arbitrary ordering or designation that cannot be reversed. Accordingly, the structure and functional properties of the first component second and/or third components disclosed herein could be those of a third component, a second component and/or a first component, respectively. Similarly, the structure and functional properties of the first binding moiety and the second binding moiety disclosed herein could be those of a second binding moiety and a first binding moiety, respectively. It will also be appreciated that the biosensor may further comprise one or more other, non-stated molecular components.

In this context, a “component” or “molecular component’ is a discrete molecule that forms a separate part, portion or component of the biosensor. In typical embodiments, each molecular component is, or comprises, a single, contiguous amino acid sequence (i.e a fusion protein). While it will be apparent that in many embodiments the first and second components may non-covalently bind, couple, interact or associate in the context of a “binding event” mediated by respective binding moieties, they remain discrete molecules that form the biosensor.

In some embodiments, the target molecule is an enzyme such as □ amylase. In such embodiments, the first and second binding moieties are, respectively the camelid antibodies VHH1 and VHH2.

In some embodiments, the target molecule is a small organic molecule such as rapamycin. In such embodiments, the first and second binding moieties are, respectively the FKBP and FRB. Non-limiting examples of particular forms of this general embodiment are generally shown in FIGS. 5A and 6.

In some embodiments, the target molecule is a small organic molecule such as FK506. In such embodiments, the first and second binding moieties are, respectively, the FKBP and a Calcineurin A/B complex, such as shown in FIG. 5B.

In some embodiments, the target molecule is a small organic molecule such as cyclosporin. In such embodiments, the first and second binding moieties are, respectively, a peptidyl prolyl cis trans isomerase A and Calcieurin A/B complex such as shown in FIG. 5C.

In another broad embodiment, said engineered amino acid sequence of said enzyme and said at least one amino acid sequence of the enzyme capable of reacting with a substrate molecule comprise respective binding moieties that initially interact, which interaction is subsequently disrupted by one or the other of the binding moieties binding a target molecule. This disruption of this interaction facilitates the replacement of the engineered amino acid sequence by said yet another amino acid sequence.

Another particular embodiment of the second aspect therefore provides a biosensor comprising a first component comprising: at least one amino acid sequence of an enzyme capable of reacting with a substrate molecule when in a catalytically active state to produce one or more electrons; a first binding moiety; and at least one other amino acid sequence of said enzyme which is engineered to releasably maintain the enzyme in a catalytically inactive state; and a second component comprising at least one other amino acid sequence of said enzyme and a second binding moiety; arranged so that an interaction between said first and second binding moieties is released by a target molecule capable of binding the first or second binding moiety to facilitate replacement of said at least one other amino acid sequence of the first molecule by said at least one other amino acid sequence of the second molecule, to thereby switch the enzyme of the first component from a catalytically inactive state to a catalytically active state.

Preferably, the biosensor further comprises a cross-binder which initially interacts with the first and second binding moieties to releasably maintain the interaction between said first and second binding moieties. Suitably, the cross-binder is displaceable from the first and/or second binding moieties by the target molecule to thereby facilitate replacement of said at least one other amino acid sequence of the first molecule by said at least one other amino acid sequence of the second molecule.

In some embodiments, the target molecule is an illicit drug such as THC. In such embodiments, the binding moieties are a THC calmodulin binding peptide conjugate or alternatively a peptide that competitively binds the THC binding site of an anti-THC antibody fused to a calmodulin binding peptide. According to this embodiment, the cross-binder is a calmodulin binding peptide comprising or consisting of the amino acid sequence GVMPREETDSKTASPWKSARLMVHTVATFNSIKELNERWRSLQQLA (SEQ ID NO:13).

A non-limiting example of such an embodiment is shown in FIG. 9.

In another broad embodiment, the biosensor of the second aspect is suitable for detecting a protease target molecule. Preferably, said at least one amino acid sequence of the enzyme capable of reacting with a substrate and said yet another amino acid sequence of the enzyme comprise respective binding moieties that can interact after protease cleavage of an inhibitor of binding between these. This interaction facilitates the replacement of the engineered amino acid sequence by said yet another amino acid sequence.

Yet another particular embodiment of the second aspect therefore provides a biosensor comprising a first component comprising: at least one an amino acid sequence of an enzyme capable of reacting with a substrate molecule when in a catalytically active state to produce one or more electrons; a first binding moiety; and at least one other amino acid sequence of said enzyme which is engineered to releasably maintain the enzyme in a catalytically inactive state; and a second component comprising at least one other amino acid sequence of said enzyme and a second binding moiety linked or connected to an inhibitor by a protease cleavage site, wherein the inhibitor prevents or inhibits an interaction between the first and second binding moieties; arranged so that said inhibitor is released by a protease target molecule cleaving said protease cleavage site to facilitate an interaction between the first and second binding moieties to facilitate replacement of said at least one other amino acid sequence of the first molecule by said at least one other amino acid sequence of the second molecule, to thereby switch the enzyme of the first component from a catalytically inactive state to a catalytically active state.

A “protease” is a protein which displays, or is capable of displaying, an ability to hydrolyse or otherwise cleave a peptide bond. Like terms include “proteinase” and “peptidase”. Proteases include serine proteases, cysteine proteases, metalloproteases, threonine proteases, aspartate proteases, glutamic acid proteases, acid proteases, neutral proteases, alkaline proteases, exoproteases, aminopeptidases and endopeptidases although without limitation thereto. Proteases may be purified or synthetic (e.g. recombinant synthetic) forms of naturally-occurring proteases or may be engineered or modified proteases which comprise one or more fragments or domains of naturally-occurring proteases which, optionally, have been further modified to possess one or more desired characteristics, activities or properties.

The target protease may be any protease for which a protease cleavage site is known. Suitably, the target protease is detectable in a biological sample obtainable from an organism, inclusive of bacteria, plants and animals. Animals may include humans and other mammals. Non-limiting examples of target proteases include proteases involved in blood coagulation such as thrombin, plasmin, factor VII, factor IX, factor X, factor Xa, factor XI, factor XII (Hageman factor) and other proteases such as kallikreins (e.g. kallikrein III, P-30 or prostate specific antigen), matrix metalloproteinases (such as involved in wounds and ulcers; e.g. MMP7 and MMP9), adamalysins, serralysins, astacins and other proteases of the metzincin superfamily, trypsin, chymotrypsin, elastase, cathepsin G, pepsin and carboxypeptidase A as well as proteases of pathogenic viruses such as HIV protease, West Nile NS3 protease and dengue virus protease although without limitation thereto.

A non-limiting example of this embodiment is shown in FIG. 8.

In a third aspect, the invention provides a biosensor molecule comprising at least one amino acid sequence of an enzyme capable of reacting with a substrate molecule when in a catalytically active state to produce one or more electrons; a binding moiety capable of binding a target molecule; and at least one enzyme inhibitor which is capable of interacting with the binding moiety in the absence of the target molecule to thereby inhibit the enzyme; arranged so that the target molecule can release the interaction between said at least one enzyme inhibitor and the binding moiety to thereby release inhibition of the enzyme by the inhibitor and switch the amino acid sequence of the enzyme from a catalytically inactive state to said catalytically active state.

The enzyme, the substrate molecule, the target molecule and/or the binding moiety may be any enzyme, substrate molecule, target molecule and/or binding moiety as hereinbefore described.

A particular feature of this aspect is that the binding moiety can bind or interact with the target molecule, if present. In a preferred initial state, the binding moiety releasably interacts with or binds the enzyme inhibitor, or a molecule (e.g an amino acid sequence) linked to the enzyme inhibitor. This facilitates enzyme inhibition by the enzyme inhibitor. Subsequently, if present the target molecule competes with the enzyme inhibitor, or the molecule linked thereto, thereby releasing the enzyme inhibitor from the binding moiety which results in release of enzyme inhibition, thereby switching the enzyme to a catalytically active state.

The enzyme inhibitor may be any molecule which is capable of preventing, inhibiting, preventing or suppressing the ability of the enzyme to react with the substrate molecule to provide one or more electrons.

An embodiment of the third aspect provides a biosensor comprising a first component comprising: at least one amino acid sequence of an enzyme capable of reacting with a substrate molecule when in a catalytically active state to produce one or more electrons; an inhibitor of said enzyme linked or coupled to the enzyme by a protease cleavage site; and a first component binding moiety; a second component comprising a second component binding moiety capable of binding the first component binding moiety; a protease amino acid sequence; and another second component binding moiety capable of binding a target molecule; and a third component comprising a third component binding moiety that can interact with said second component binding moieties in the absence of the target molecule; arranged so that said target molecule can displace binding between the third component binding moiety and said second component binding moieties to facilitate an interaction between said first component binding moiety and said second component binding moiety whereby the protease cleaves the protease cleavage site to remove inhibition of the enzyme by the inhibitor and thereby switch the enzyme from a catalytically inactive state to a catalytically active state.

The enzyme, the substrate molecule, the target molecule and/or the binding moieties may be any enzyme, substrate molecule, target molecule and/or binding moiety as hereinbefore described.

A particular feature of this embodiment is that the protease cleavage site of the first component of the first component is cleavable by the protease amino acid sequence of the second component. Preferably, the third component binding moiety comprises first and second portions, wherein the first portion can initially, releasably bind or interact with said second component binding moiety and the second portion can initially, releasably bind or interact with said another second component binding moiety in the absence of the target molecule. If present, the target molecule can displace the second portion of the third component from said another second component binding moiety. This facilitates release of the first portion of the third component from interacting with or binding said second component binding moiety. This facilitates the first component binding moiety binding or interacting with said second component binding moiety, thereby co-localizing the first and second components and bringing the protease amino acid sequence of the second component into the proximity of the protease cleavage site of the first component. The protease may subsequently cleave the protease cleavage site, thereby releasing the enzyme from the enzyme inhibitor, which results in release of enzyme inhibition, thereby switching the enzyme to a catalytically active state.

In a related aspect, the invention further provides an oxidoreductase enzyme, preferably a GDH enzyme, comprising an inhibitory moiety acting to prevent or reduce catalytic activity of the enzyme, wherein the inhibitory moiety can be displaced in the presence of one or more molecules to activate catalytic activity of the enzyme. The inhibitory moiety is also described herein as an enzyme inhibitor and may be any molecule capable of preventing or reducing catalytic activity of the enzyme, which is able to be displaced to activate catalytic activity.

The inhibitory moiety may displace catalytic amino acid residues at the active site. The inhibitory moiety may sterically prevent access of substrate to the active site. The inhibitory moiety is preferably an antibody or antibody fragment or an inhibitory peptide. The inhibitory moiety may be located N- or C-terminally at the enzyme, including at the N- or C-terminus of the enzyme, or may be located internally in the amino acid sequence of the enzyme. The enzyme may comprise one or more mutations increasing the ability of the inhibitory moiety (such as an inhibitory peptide) to prevent or reduce catalytic activity of the enzyme, for example by increasing affinity of the inhibitory peptide for a region within the enzyme such that it binds or anchors more strongly to said region, and thereby prevents or reduces catalytic activity.

In embodiments relating to GDH enzymes, the inhibitory moiety may be an antibody or antibody fragment or inhibitory peptide located at the C-terminus of the enzyme. The GDH enzyme may comprise the sequence of SEQ ID NO:1 or a variant thereof. The inhibitory moiety may comprise the sequence of any of SEQ ID Nos 20-24, 51 or 55 or a variant thereof, which is typically added in the C-terminal region of said GDH enzyme, such as at or close to the C-terminus.

Where the GDH enzyme is a mutant enzyme as described above, comprising amino acid mutations assisting binding of the inhibitory moiety, it typically comprise such mutations at one or more positions corresponding to positions 340-344 of SEQ ID NO: 1 (EMTY1 in the native PQQ-GDH enzyme of SEQ ID NO: 1) which are able to enhance inhibitory activity of an inhibitory moiety located at the C-terminus of the enzyme. The variant preferably retains the tyrosine residue at position 343 of SEQ ID NO:1. The variant may comprise mutations at one or more of positions 340 to 342 and 344 which introduce polar or hydrophilic amino acid residues. The variant may comprise the sequence SSSYS (SEQ ID NO: 51) or a variant thereof at positions corresponding to positions 340-344 of SEQ ID NO: 1. The mutant enzyme may comprise the sequence of SEQ ID NO: 31 or a variant thereof. Representative examples of inhibitory moieties that may be added to a mutant GDH enzyme as described above (such as a GHD enzyme comprising the sequence of SEQ ID NO: 31 or a variant thereof) include SEQ ID Nos 25-30 or variants thereof. These inhibitory moieties are typically included C-terminally in the enzyme, as described above. Representative examples of mutant GDH enzymes incorporating inhibitory moieties (autoinhibited enzymes) are provided by SEQ ID Nos 31 to 33 and 54. The invention provides autoinhibited mutant GDH enzymes comprising the sequence of any of SEQ ID Nos 31 to 33 and 54 or variants thereof.

The above-described oxidoreductase (such as GDH) enzyme typically comprises one or more protease cleavage sites, wherein cleavage of a said site by a protease displaces the inhibitory moiety to activate catalytic activity of the enzyme. The enzyme may further comprise a sequence enhancing binding and/or cleavage efficiency of the protease.

The oxidoreductase enzyme may comprise a binding moiety capable of interacting with a respective binding moiety on a further molecule, wherein interaction between the binding moieties displaces the inhibitory moiety to activate catalytic activity of the enzyme. Such an oxidoreductase enzyme may further comprise one or more protease cleavage sites, wherein the further molecule additionally comprises a protease and interaction between the binding moieties acts to bring the protease into proximity with a said site to cleave said site and displace the inhibitory moiety. The binding moieties and protease cleavage site(s) may be selected from any of those described herein.

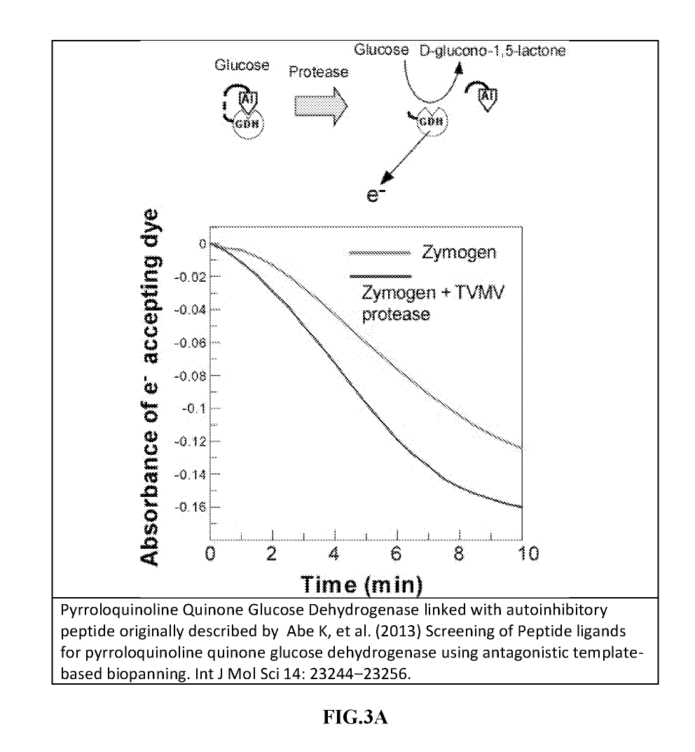

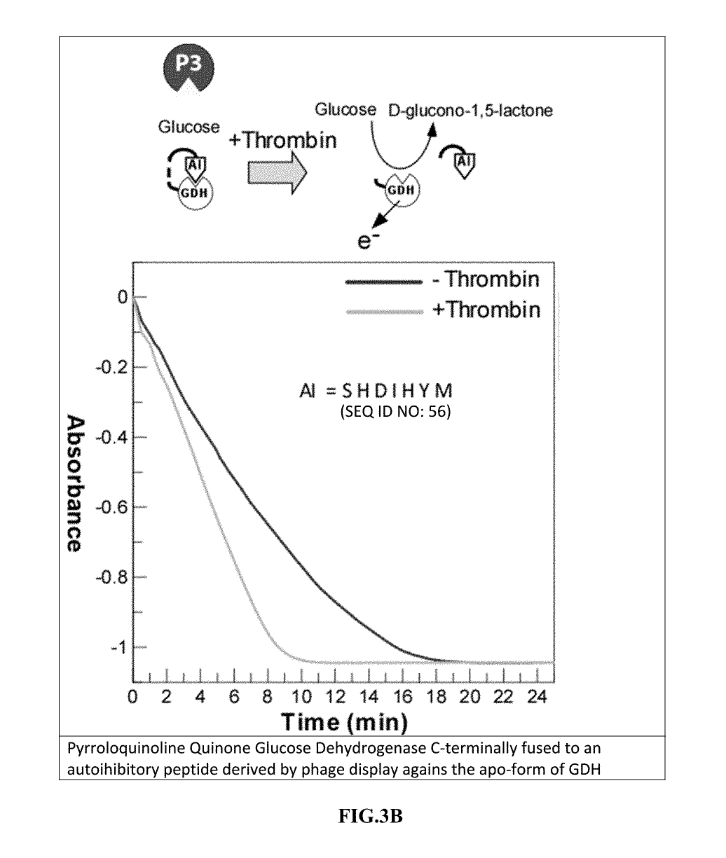

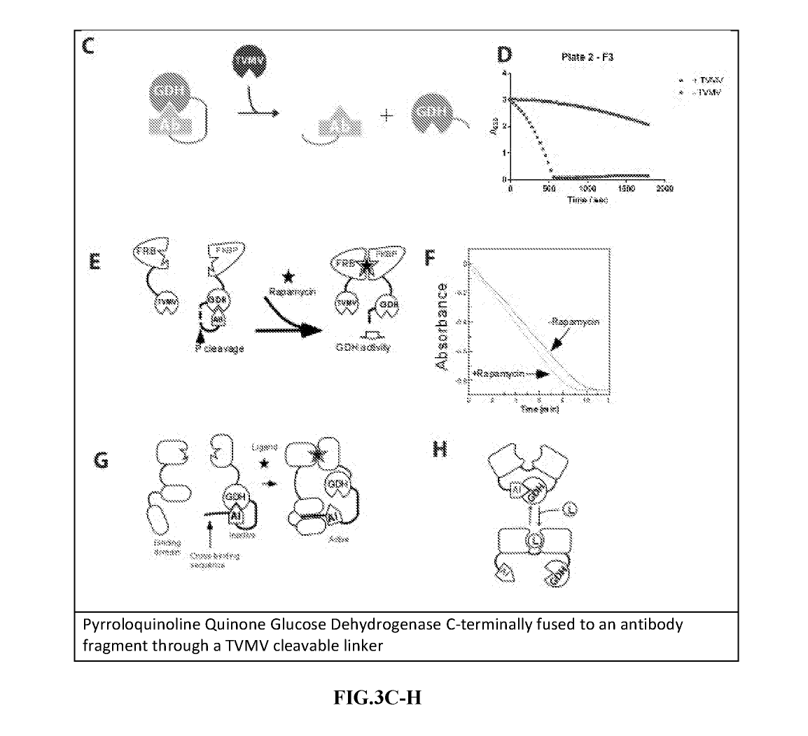

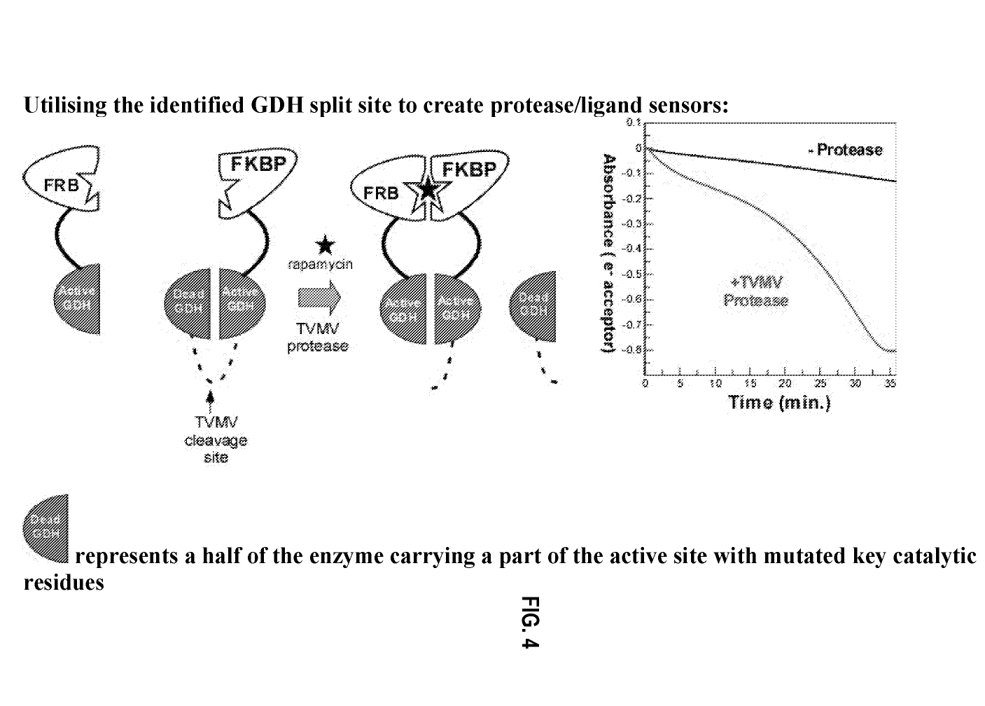

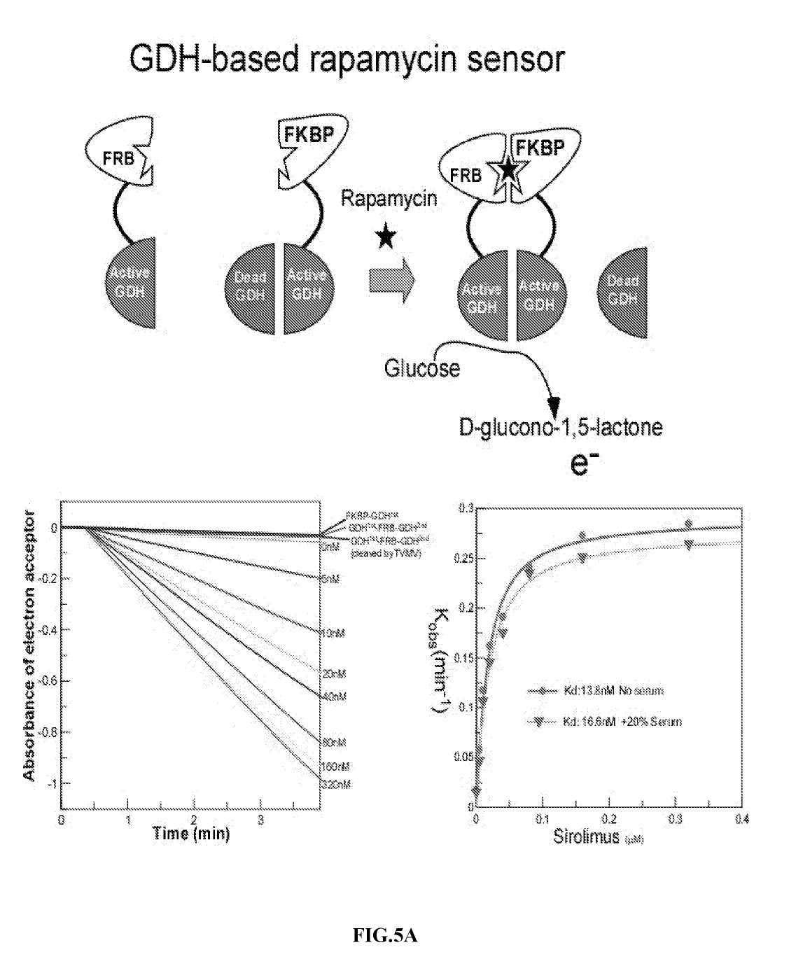

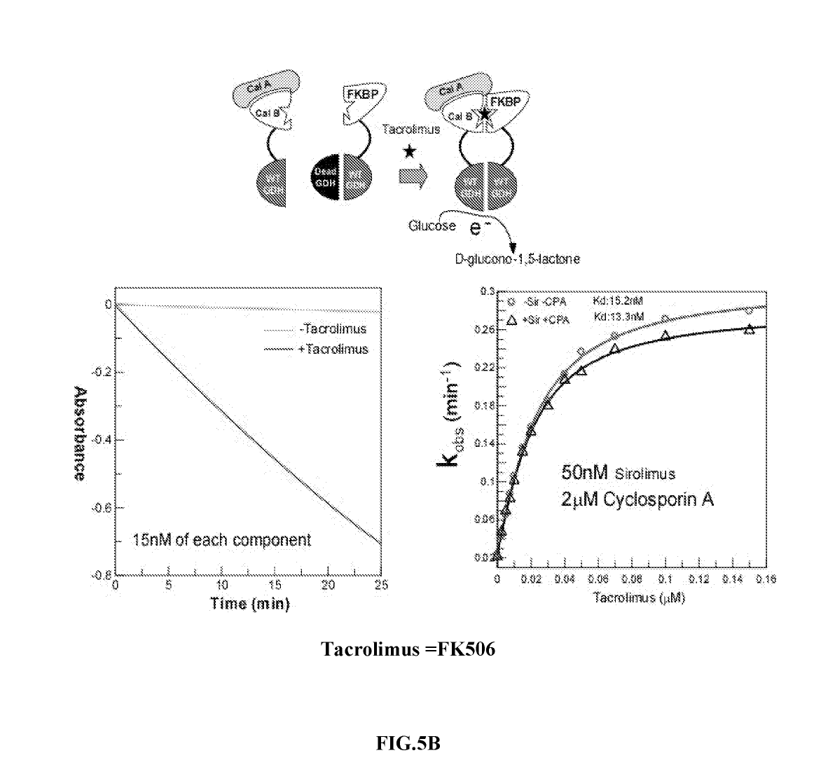

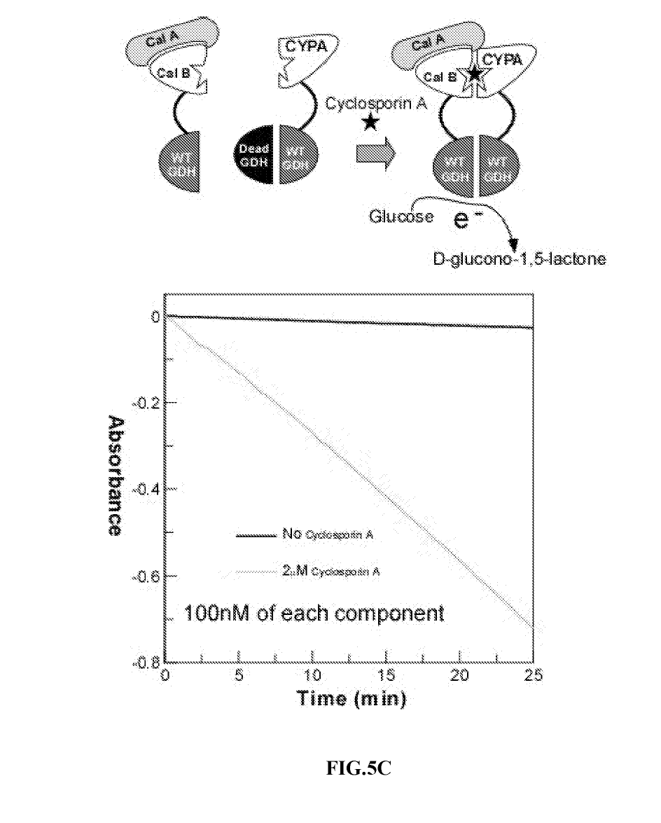

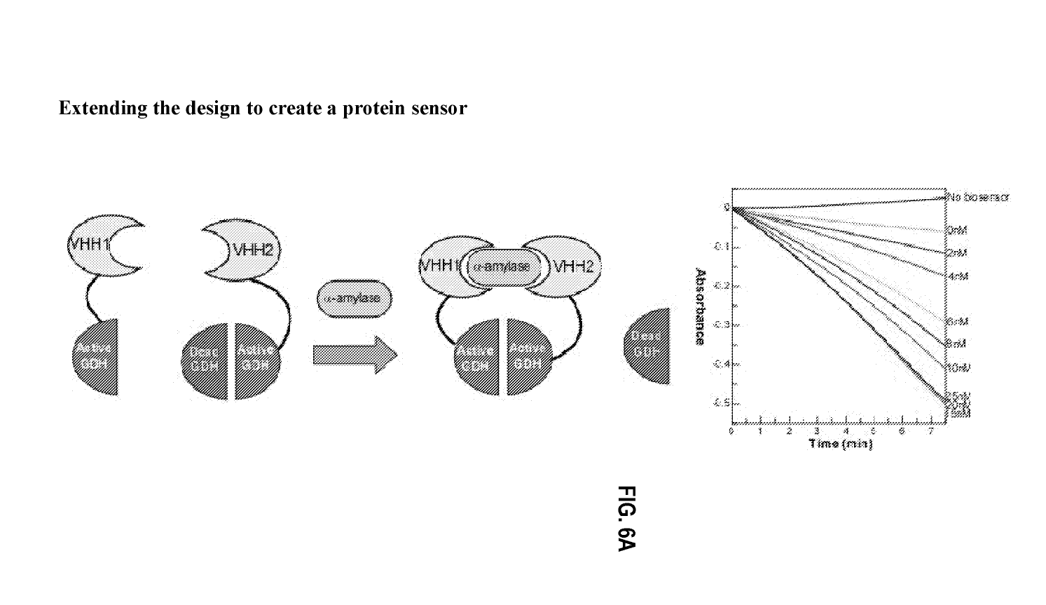

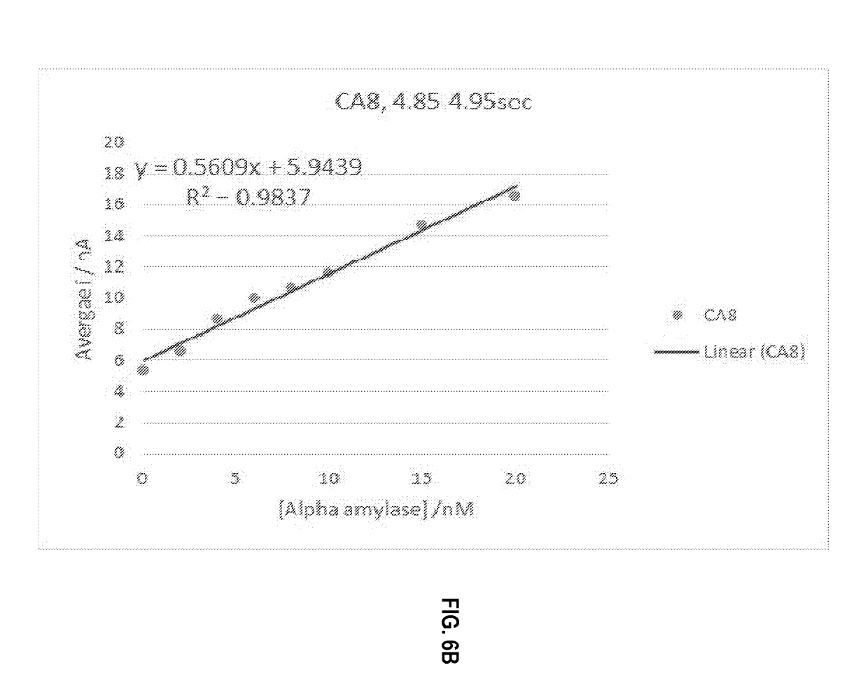

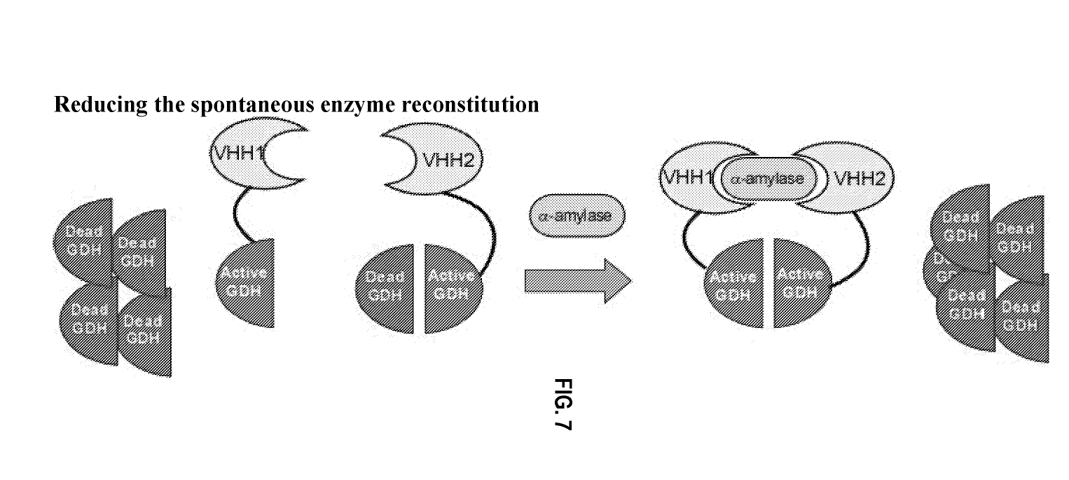

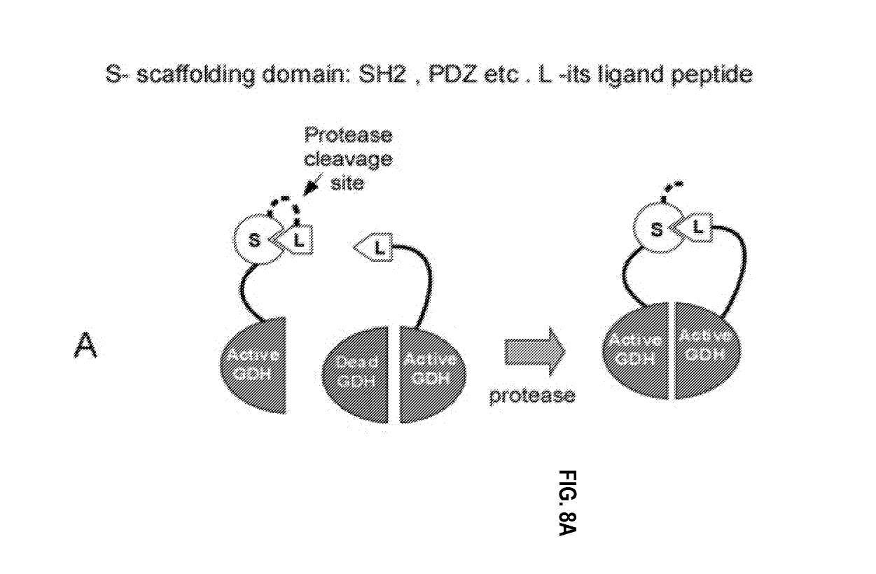

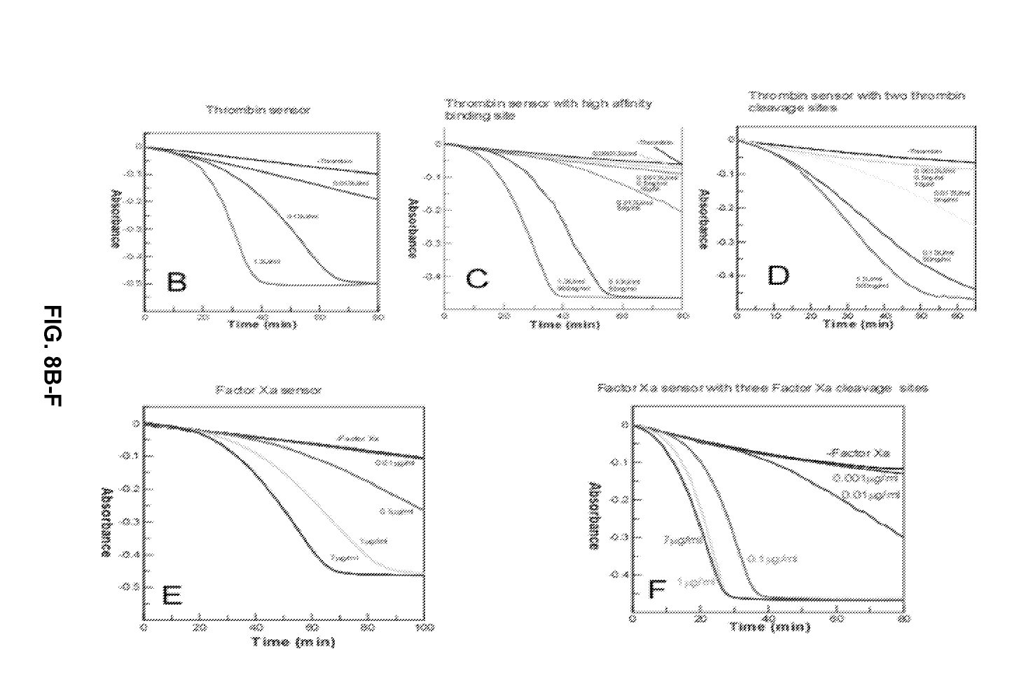

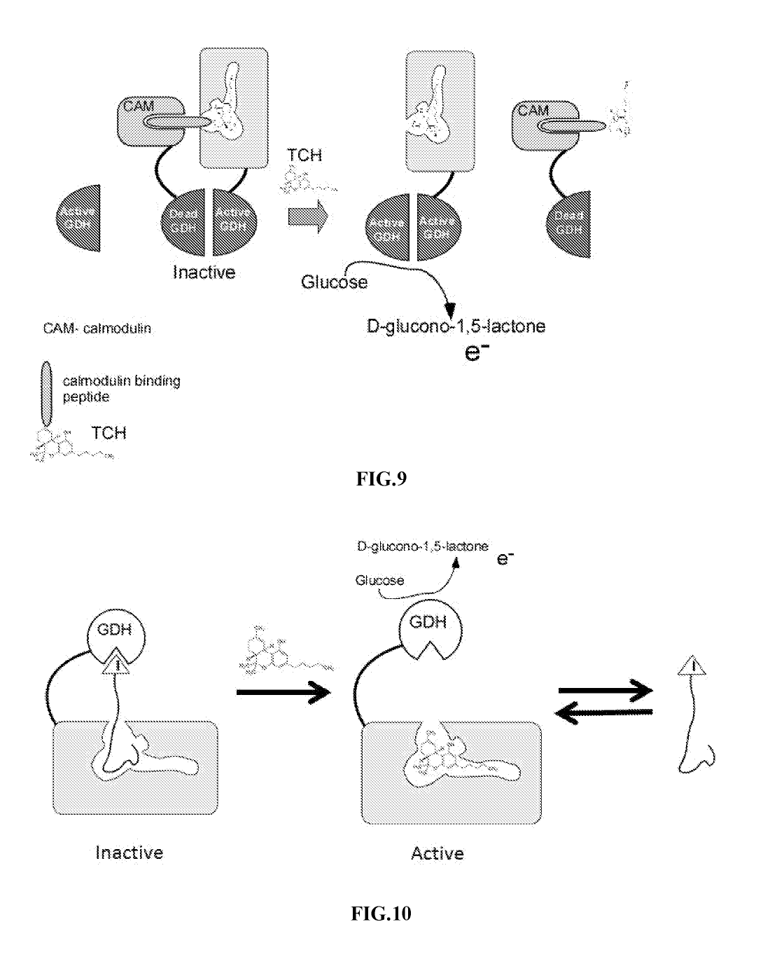

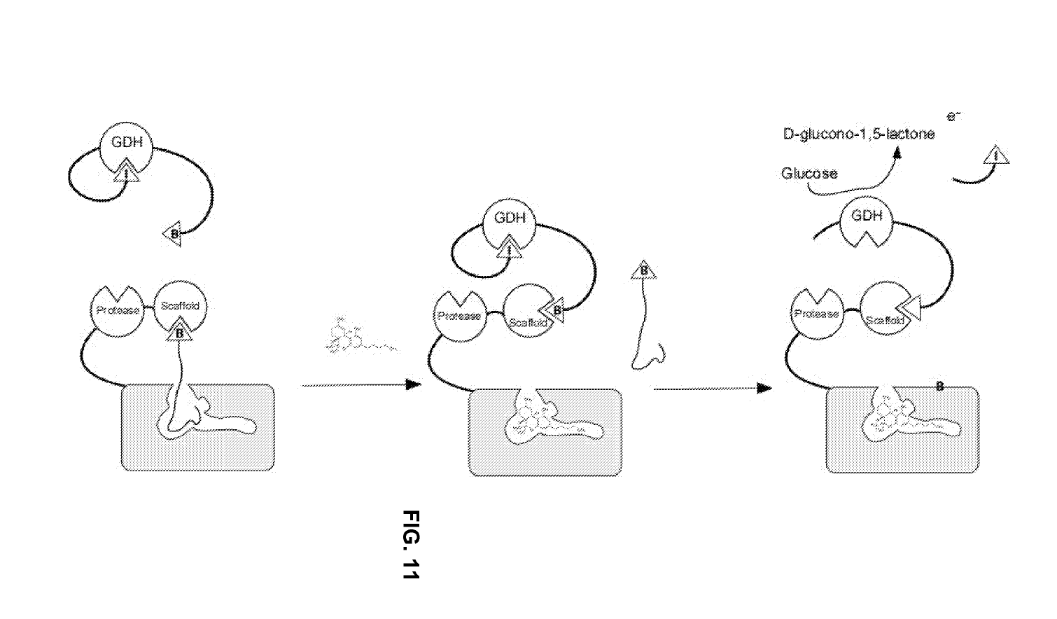

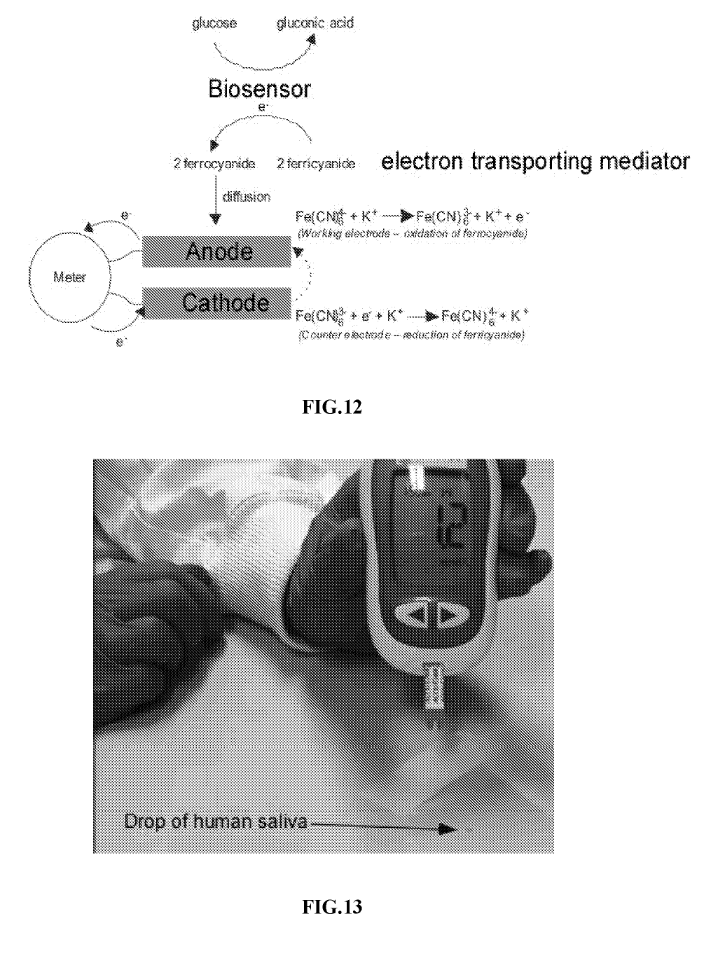

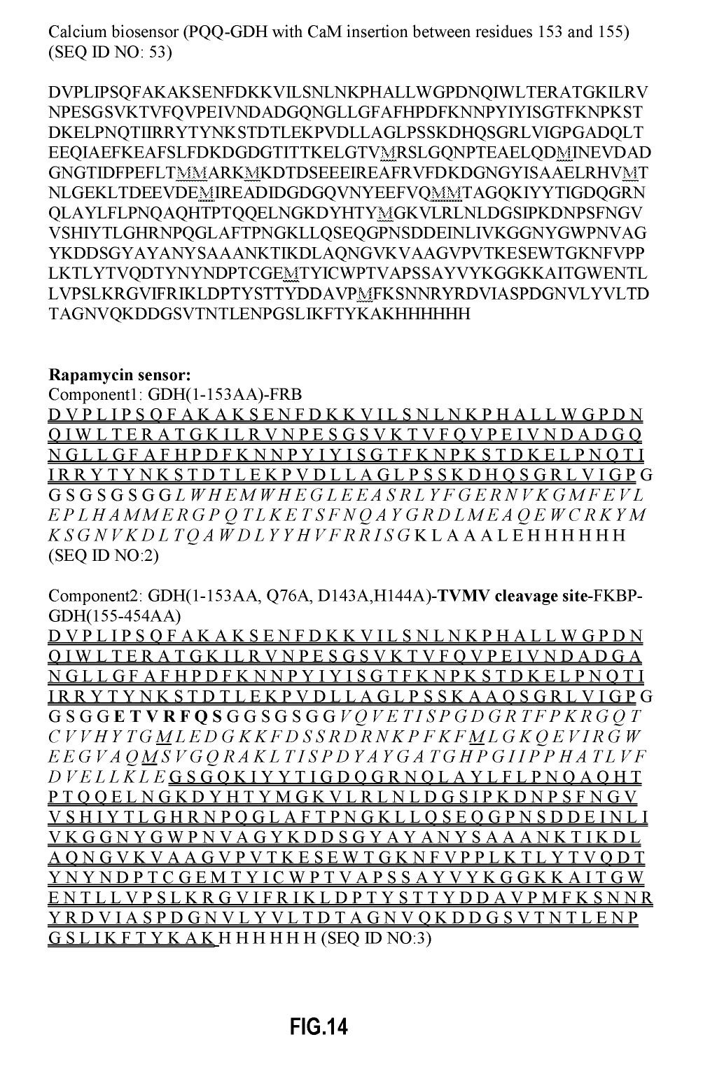

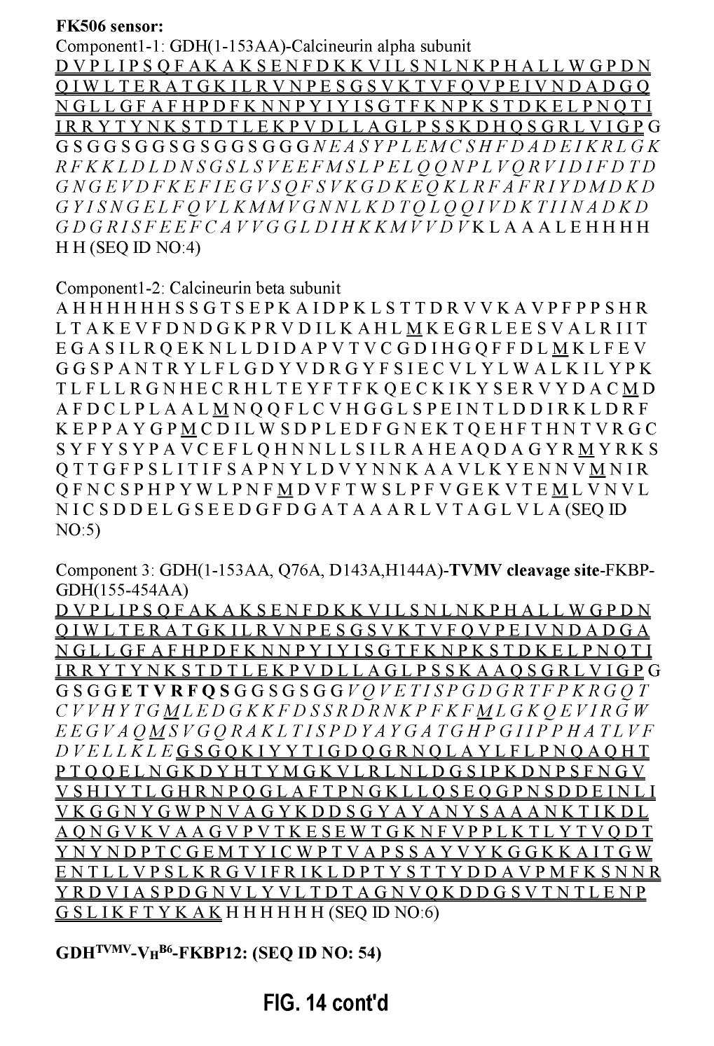

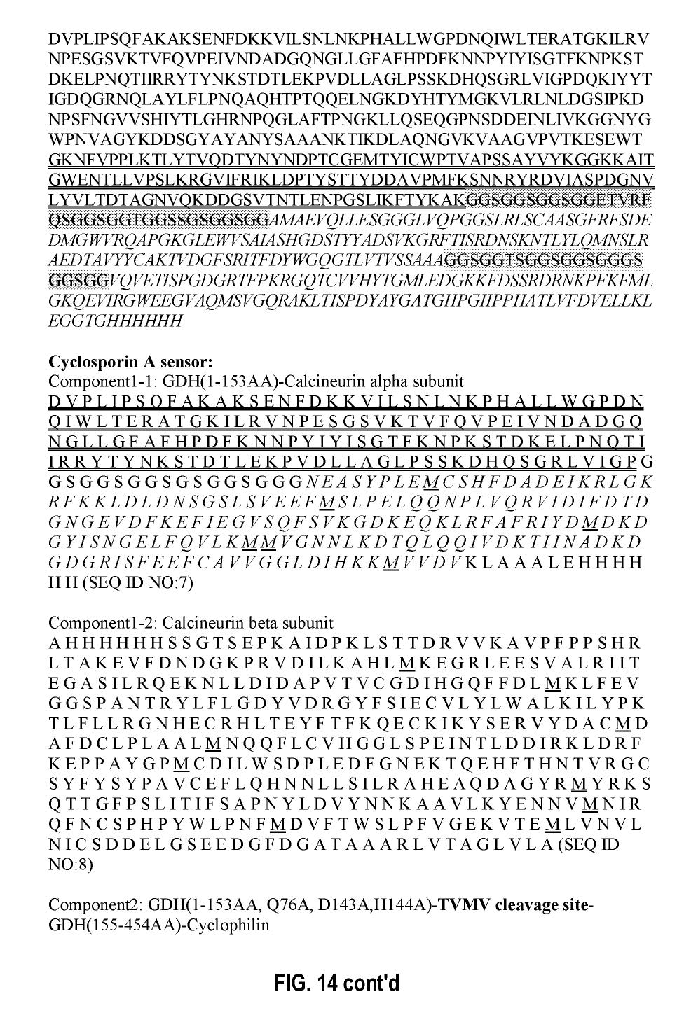

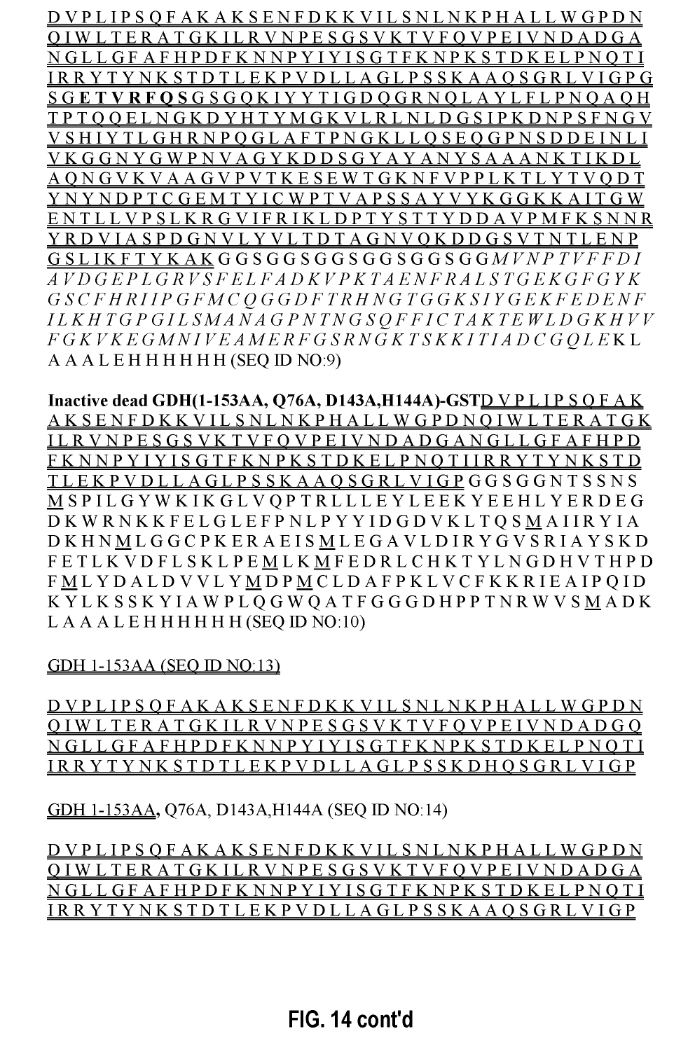

Examples of autoinhibited GDH enzymes activated by protease cleavage are shown in FIGS. 3A-E.

The invention further provides a method of engineering an autoinhibited oxidoreductase (such as GDH) enzyme, comprising screening for an inhibitory moiety able to prevent or reduce catalytic activity when fused to said enzyme, wherein the inhibitory moiety is able to be displaced in the presence of a target molecule to reconstitute catalytic activity. The inhibitory moiety may be incorporated N- or C-terminally in the sequence of the enzyme. In embodiments relating to GDH enzymes, the inhibitory moiety is preferably provided C-terminally in the enzyme, such as fused to the C-terminus. The GHD enzyme may comprise the sequence of SEQ ID NO: 1 or SEQ ID NO: 31 or a variant of either thereof. A putative inhibitory moiety may be identified by phage display. The screening may be carried out in an in vitro activity assay. A suitable assay is described in the Examples herein.

The protease amino acid sequence may be an entire amino acid sequence of a protease or may be an amino acid sequence of a proteolytically-active fragment or sub-sequence of a protease.

In some embodiments, the protease may be an autoinhibited protease.

In one preferred embodiment, the protease is an endopeptidase.

In some embodiments, proteases are derived from, or encoded by, a viral genome. Typically, such proteases are dependent on expression and proteolytic processing of a polyprotein and/or other events required as part of the life cycle of viruses such as Picornavirales, Nidovirales, Herpesvirales, Retroviruses and Adenoviruses, although without limitation thereto. Particular examples of proteases include: Potyviridae proteases such as the NIa protease of tobacco etch virus (TEV), tobacco vein mottling virus (TVMV), sugarcane mosaic virus (SMV) etc; Flaviviridae proteases such as the NS3 protease of hepatitis C virus (HCV); Picornaviridae proteases such as the 3C protease of EV71, Norovirus etc, the 2A protease of human rhinovirus, coxsackievirus B4 etc and the leader protease of foot and mouth disease virus (FMDV) etc; Coronaviridae proteases such as the 3C-like protease of SARS-CoV, IBV-CoV and Herpesvirus proteases such as HSV-1, HSV-2, HCMV and MCMV proteases etc, although without limitation thereto.

Preferably, the viral genome is of a plant virus.

More preferably, the plant virus is a Potyvirus.

In a particularly preferred embodiment, the protease is a Potyvirus protease such as the NIa protease of TEV, TVMV or SMV.

In an alternative embodiment the protease is an NS3 protease of a Flavivirus such as HCV.

In other embodiments, proteases are SUMO related proteases that includes ubiquitin (Ub), NEDD8, and Atg 8 proteases^{[21]}. These proteases are converted into an autoinhibited form by fusion with their respective recognition domains (e.g SUMO) via a protease-resistant linker.

In one particular embodiment, the second component binding moiety is a scaffolding protein, domain or fragment thereof. Non-limiting examples include a PDZ domain, an SH2 or SH3 domain, or a fragment thereof. As previously described, preferably the third component binding moiety comprises first and second portions, wherein the first portion can initially, releasably bind or interact with said second component binding moiety and the second portion can initially, releasably bind or interact with said another second component binding moiety in the absence of the target molecule. The first portion may be a ligand (such as a peptide) which binds the scaffolding protein, domain or fragment thereof.

The second portion of the third component may be a peptide binding competitively to the binding site of the target molecule or the target molecule or an analogue thereof conjugated to the first portion.

Non-limiting examples of these embodiments are shown in FIGS. 10 and 11.

It will be appreciated that the biosensors and the molecular components thereof described herein may be, or comprise, contiguous amino acid sequences such as in the form of chimeric proteins or fusion proteins as are well understood in the art. Optionally, respective amino acid sequences (e.g binding moieties, enzyme amino acid sequences, protease amino acid sequences etc) may be discrete or separate amino acid sequences linked or connected by spacers or linkers (e.g. amino acids, amino acid sequences, nucleotides, nucleotide sequences or other molecules) to optimize features or activities such as target molecule recognition, binding and enzyme activity or inhibition, although without limitation thereto. Non-limiting examples of amino acid sequences inclusive of enzyme amino acid sequences, engineered mutants, linkers, protease cleavage sites, and binding moieties are provided in FIG. 14 and SEQ ID NOS:1-55.

It will also be appreciated that the invention includes biosensor molecules that are variants of the embodiments described herein, or which comprise variants of the constituent protease, sensor and/or inhibitor amino acid sequences disclosed herein. Typically, such variants have at least 80%, at least 85%, preferably at least 90%, 91%, 92%, 93%, 94% 95%, 96%, 97%, 98% or 99% sequence identity with any of the amino acid sequences disclosed herein, such as SEQ ID NOS:1-55 or portions thereof. By way of example only, conservative amino acid variations may be made without an appreciable or substantial change in function. For example, conservative amino acid substitutions may be tolerated where charge, hydrophilicity, hydrophobicity, side chain “bulk”, secondary and/or tertiary structure (e.g. helicity), target molecule binding, protease activity and/or protease inhibitory activity are substantially unaltered or are altered to a degree that does not appreciably or substantially compromise the function of the biosensor. Variants of the invention (other than the engineered non-active mutants described herein) are selected to be functional and so retain or substantially retain catalytic activity, or the ability to reconstitute such catalytic activity when provided together with suitable further components of a biosensor as described above. Variants of the non-covalently associating amino acid sequences (such as first and second fragment sequences) described herein are selected to retain the ability to reconstitute a stable enzyme when provided in combination with their respective binding partner sequence.

The term “sequence identity” is used herein in its broadest sense to include the number of exact amino acid matches having regard to an appropriate alignment using a standard algorithm, having regard to the extent that sequences are identical over a window of comparison. Sequence identity may be determined using computer algorithms such as GAP, BESTFIT, FASTA and the BLAST family of programs as for example disclosed by Altschul et al., 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

Protein fragments may comprise up to 5%, 10%, 15%, 20%, 25%, 30%, 35%, 40%, 45%, 50%, 55%, 60%, 65%, 70%, 75%, preferably up to 80%, 85%, more preferably up to 90% or up to 95-99% of an amino acid sequence disclosed herein. In some embodiments, the protein fragment may comprise up to 5, 10, 20, 40, 50, 70, 80, 90, 100, 120, 150, 180 200, 220, 230. 250, 280, 300, 330, 350, 400 or 450 amino acids of an amino acid sequence disclosed herein, such as SEQ ID NOS:1-55.

As will be understood from the foregoing, the biosensors described herein produce electrons by reacting with substrate molecules in response to binding, interacting with or otherwise detecting one or more target molecules. In this context “react”, “reaction” or “reacting” with a substrate molecule means enzymatically transforming the substrate molecule into one or more product molecules with a net or overall production of one or a plurality of electrons per substrate molecule. Accordingly, the biosensor acts as an electron donor, whereby the electrons produced by the reaction may flow either directly or via an electron shuttle such as, but not limited to, phenazine methosulfate or potassium ferrocyanide, to thereby act as an anode. The resulting change in potential between anode and cathode is detected by an electronic detector. One non-limiting example of such arrangement is shown in the FIG. 12.

A further aspect of the invention provides a kit or composition comprising one or more biosensors disclosed herein in combination with one or more substrate molecules.

In a further aspect, the invention provides a method of detecting a target molecule, said method including the step of contacting the composition of the aforementioned aspect with a sample to thereby determine the presence or absence of the target molecule in the sample.

Suitably, the sample is a biological sample. Biological samples may include organ samples, tissue samples, cellular samples, fluid samples or any other sample obtainable, obtained, derivable or derived from an organism or a component of the organism. The biological sample can comprise a fermentation medium, feedstock or food product such as for example, but not limited to, dairy products.

In particular embodiments, the biological sample is obtainable from a mammal, preferably a human. By way of example, the biological sample may be a fluid sample such as blood, serum, plasma, urine, saliva, tears, sweat, cerebrospinal fluid or amniotic fluid, a tissue sample such as a tissue or organ biopsy or may be a cellular sample such as a sample comprising red blood cells, lymphocytes, tumour cells or skin cells, although without limitation thereto. A particular type of biological sample is a pathology sample.

Suitably, the enzyme activity of the biosensor is not substantially inhibited by components of the sample (e.g. serum proteins, metabolites, cells, cellular debris and components, naturally-occurring protease inhibitors etc).

In one embodiment, the biosensor and/or methods of use may be applicable to drug testing such as for detecting the use of illicit drugs of addiction (e.g cannabinoids, amphetamines, cocaine, heroin etc.) and/or for the detection of performance-enhancing substances in sport and/or masking agents that are typically used to avoid detection of performance-enhancing substances. This may be applicable to the detection of banned performance-enhancing substances in humans and/or other mammals such as racehorses and greyhounds that may be subjected to illicit “doping” to enhance performance.

In another particular embodiment, the biosensor and/or methods of use are for diagnosis of a disease or condition of a mammal, such as a human.

Accordingly, a preferred aspect of the invention provides a method of diagnosis of a disease or condition in a human, said method including the step of contacting the composition of the aforementioned aspect with a biological sample obtained from the human to thereby determine the presence or absence of a target molecule in the biological sample, determination of the presence or absence of the target molecule facilitating diagnosis of the disease or condition.

The disease or condition may be any where detection of a target molecule assists diagnosis. Non limiting examples of target molecules or analytes include blood coagulation factors such as previously described, kallikreins inclusive of PSA, matrix metalloproteinases, viral and bacterial proteases, antibodies, glucose, triglycerides, lipoproteins, cholesterol, tumour antigens, lymphocyte antigens, autoantigens and autoantibodies, drugs, salts, creatinine, blood serum or plasma proteins, pesticides, uric acid, products and intermediates of human and animal metabolism and metals.

This preferred aspect of the invention may be adapted to be performed as a “point of care” method whereby determination of the presence or absence of the target molecule may occur at a patient location which is then either analysed at that location or transmitted to a remote location for diagnosis of the disease or condition.

A still yet further aspect of the invention provides a detection device that comprises a cell or chamber that comprises the biosensor of any of the aforementioned aspects.

Suitably, a sample may be introduced into the cell or chamber to thereby facilitate detection of a target molecule.

In certain embodiments, the detection device is capable of providing an electrochemical, acoustic and/or optical signal that indicates the presence of the target molecule.

The detection device may further provide a disease diagnosis from a diagnostic target result by comprising:
a processor; and
a memory coupled to the processor, the memory including computer readable program code components that, when executed by the processor, perform a set of functions including:
analysing a diagnostic test result and providing a diagnosis of
the disease or condition.

The detection device may further provide for communicating a diagnostic test result by comprising:
a processor; and
a memory coupled to the processor, the memory including computer readable program code components that, when executed by the processor, perform a set of functions including:
transmitting a diagnostic result to a receiving device; and
optionally receiving a diagnosis of the disease or condition from the or another receiving device.

Diagnostic aspects of the invention may also be in the form of a kit comprising one or a plurality of different biosensors capable of detecting one or a plurality of different target molecules. In this regard, a kit may comprise an array of different biosensors capable of detecting a plurality of different target molecules. The kit may further comprise one or more amplifier molecules, deactivating molecules and/or labeled substrates, as hereinbefore described. The kit may also comprise additional components including reagents such as buffers and diluents, reaction vessels and instructions for use.

A further aspect of the invention provides an isolated nucleic acid which encodes an amino acid sequence of the biosensor of the invention, or a variant thereof as hereinbefore defined.

The term “nucleic acid” as used herein designates single- or double-stranded mRNA, RNA, cRNA, RNAi, siRNA and DNA inclusive of cDNA, mitochondrial DNA (mtDNA) and genomic DNA.

A “polynucleotide” is a nucleic acid having eighty (80) or more contiguous nucleotides, while an “oligonucleotide” has less than eighty (80) contiguous nucleotides. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

The invention also provides variants and/or fragments of the isolated nucleic acids. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence disclosed herein. In other embodiments, nucleic acid variants may hybridize with the nucleotide sequence of with any nucleotide sequence disclosed herein, under high stringency conditions.

Fragments may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence disclosed herein.

Fragments may comprise or consist of up to 20, 50, 100, 150, 200, 250, 300, 350, 400, 450, 500, 550, 600, 650, 700, 750, 800, 850, 900 950, 1000, 1050, 1100, 1150, 1200, 1350 or 1300 contiguous nucleotides present in any nucleotide sequence disclosed herein.

The invention also provides “genetic constructs” that comprise one or more isolated nucleic acids, variants or fragments thereof as disclosed herein operably linked to one or more additional nucleotide sequences.

As generally used herein, a “genetic construct” is an artificially created nucleic acid that incorporates, and/or facilitates use of, an isolated nucleic acid disclosed herein.

In particular embodiments, such constructs may be useful for recombinant manipulation, propagation, amplification, homologous recombination and/or expression of said isolated nucleic acid.

As used herein, a genetic construct used for recombinant protein expression is referred to as an “expression construct”, wherein the isolated nucleic acid to be expressed is operably linked or operably connected to one or more additional nucleotide sequences in an expression vector.

An “expression vector” may be either a self-replicating extra-chromosomal vector such as a plasmid, or a vector that integrates into a host genome.

In this context, the one or more additional nucleotide sequences are regulatory nucleotide sequences.

By “operably linked” or “operably connected” is meant that said regulatory nucleotide sequence(s) is/are positioned relative to the nucleic acid to be expressed to initiate, regulate or otherwise control expression of the nucleic acid.

Regulatory nucleotide sequences will generally be appropriate for the host cell used for expression. Numerous types of appropriate expression vectors and suitable regulatory sequences are known in the art for a variety of host cells.

One or more regulatory nucleotide sequences may include, but are not limited to, promoter sequences, leader or signal sequences, ribosomal binding sites, transcriptional start and termination sequences, translational start and termination sequences, splice donor/acceptor sequences and enhancer or activator sequences.

Constitutive or inducible promoters as known in the art may be used and include, for example, nisin-inducible, tetracycline-repressible, IPTG-inducible, alcohol-inducible, acid-inducible and/or metal-inducible promoters.

In one embodiment, the expression vector comprises a selectable marker gene. Selectable markers are useful whether for the purposes of selection of transformed bacteria (such as bla, kanR, ermB and tetR) or transformed mammalian cells (such as hygromycin, G418 and puromycin resistance).

Suitable host cells for expression may be prokaryotic or eukaryotic, such as bacterial cells inclusive of Escherichia coli (DH5α for example), yeast cells such as S. cerivisiae or Pichia pastoris, insect cells such as SF9 cells utilized with a baculovirus expression system, or any of various mammalian or other animal host cells such as CHO, BHK or 293 cells, although without limitation thereto.

Introduction of expression constructs into suitable host cells may be by way of techniques including but not limited to electroporation, heat shock, calcium phosphate precipitation, DEAF dextran-mediated transfection, liposome-based transfection (e.g. lipofectin, lipofectamine), protoplast fusion, microinjection or microparticle bombardment, as are well known in the art.

Purification of the recombinant biosensor molecule may be performed by any method known in the art. In preferred embodiments, the recombinant biosensor molecule comprises a fusion partner (preferably a C-terminal His tag) which allows purification by virtue of an appropriate affinity matrix, which in the case of a His tag would be a nickel matrix or resin.

So that the invention may be readily understood and put into practical effect, embodiments of the invention will be described with reference to the following non-limiting Examples.

### EXAMPLES

### Example 1

### Engineering an Electrochemical Biosensor Based on Acinetobacter calcoaceticus Pyrroloquinoline Quinone Glucose Dehydrogenase

Over the last three decades the biosensors became a practical alternative to complex and expensive analytical instruments used in healthcare^{[1]}. Among several currently used detection technologies such optical, acoustic, piezoelectric electrochemical sensors feature prominently due to their simplicity, specificity and high performance^{[2]}. Electrochemical blood glucose sensors are the most commercially successful biosensors accounting for nearly 90% of the biosensor market^{[3]}. The success of these sensors is due to their high selectivity and sensitivity combined with the simplicity of design and the ease of manufacturing. The sensors are based on amperometric monitoring of the glucose oxidation by recombinant glucose oxidase or glucose dehydrogeneases^{[4]}. Here the dry analysis chamber is flooded with the sample setting off the enzymatic reaction with high current density. The third generation of glucose sensors is also independent of the oxygen and electron transfer mediators making the system less drift-prone^{[5]}. The simplicity and robustness of the design enables manufacturing of disposable glucose sensing electrodes for less than $0.1^{[6]}. Remarkably, this technological and commercial success has not been paralleled by other electrochemical biosensors despite both the clinical demand and the commercial potential of Point-of-Care diagnostics. This can be at least in part explained by the unique features of glucose sensing where the analyte is present at high (4-10 mM) concentration and is also provides the source of energy for a selective, physically stable and highly processive electrochemical receptor.

### Materials and Methods

### Chimeric Gene Construction and Protein Expression and Purification

The constructs of GDH-CaM chimeric proteins were generated by Gibson Assembly™ method according the manufacturer instruction (New England Biolab) and cloned into PET28a vector. The gene fragments for the assemble were made either by PCR or by Gblcok gene synthesis from IDT (Integrated DNA Technologies). The protein expression and purification were described by Olsthoorn & Duine¹⁹. The purified GDH-CaM were reconstituted by adding PQQ with 1:1.5 ratio. This ratio for reconstitution of GDH and PQQ was also used in all other experiments using PQQ-GDH enzymes described herein.

The proteins of cyclosporine sensor were purified as described previously (http://www.pnas.org/content/99/21/13522). After Ni-NTA purification the pooled enzyme-containing fractions were supplemented with EDTA to the final concentration 5 mM and dialyzed against buffer containing 20 mM KH₂PO₄ pH7.0 and 5 mM EDTA for 10 hours. Subsequently EDTA was removed by dialyzing the sample against the buffer containing 20 mM KH₂PO₄ pH7.0 only.

### Analysis of GDH Enzymatic Activity

The GDH enzyme assay was performed as described by Yu et al.²⁰ Briefly, the 1.5-mL assay system consisted of 20 mM glucose, 0.6 mM phenazine methosulfate, 0.06 mM 2,6-dichlorophenol, 10 mM MOPS (pH 7.0), and corresponding concentration of CaCl₂ and enzyme. The enzymatic assay was performed at 25° C. by monitoring the reduction in the absorbance of 2,6-dichlorophenol at 600 nm.

### Electrochemical Analysis of GDH-Cam Activity

Chonoamperometric measurements were carried out using a Digy-Ivy DY2116B 3-electrode mini-potentiostat interfaced to DropSens disposable screen printed gold electrodes (Cat#DRP-C220BT). Electrodes were washed with 98° C. milliQ water between scans to ensure no bound active enzyme was present. Reactions contained: 300 nM GDH-CaM, PMS mediator at 3 mM, glucose at 50 mM in 50 μl total volume at pH 7.6 PBS. Reactions were started with the addition of 40 mM glucose and incubated at room temperature for 1 minute before being pipetted onto the electrode surface. Chronoamperometry was carried out for 5 s at +0.4 V versus the imbedded silver strip on the screen printed electrode, with data generally reported as current at the 5 s time point versus calcium concentration. (FIG. 2C)

### Results

We conjectured that technological advances made in glucose biosensors could be explored for the design of biosensors to analytes not structurally related to glucose. To this end we chose to Acinetobacter calcoaceticus pyrroloquinoline quinone glucose dehydrogenase (PQQ-GDH) capable of direct electron transfer as the starting point for biosensor design^{[7][8]}. However, rather than seeking to modify enzyme's substrate specificity we wanted to endow the enzyme with the allosteric receptor domain that would control its catalytic activity in ligand dependent fashion. To achieve that we analyzed the high resolution structure of A. calcoaceticus PQQ-GDH (PDB: 1CQ1) for possible sites in the vicinity of active center that would be close enough to transmit the conformational changes into the active center and in the same time far enough to tolerate insertion of a receptor domain.

The PQQ-GDH amino acid sequence including the underlined N-terminal leader sequence is set forth in SEQ ID NO:50). The mature PQQ-GDH amino acid sequence with the N-terminal leader sequence cleaved is set forth in SEQ ID NO:1:

Protein sequence before cleavage of signal
sequence (SEQ ID NO: 50):
M N K H L L A K I A L L G A A Q L V T L S A F A D
V P L I P S Q F A K A K S E N F D K K V I L S N L
N K P H A L L W G P D N Q I W L T E R A T G K I L
R V N P E S G S V K T V F Q V P E I V N D A D G Q
N G L L G F A F H P D F K N N P Y I Y I S G T F K
N P K S T D K E L P N Q T I I R R Y T Y N K S T D
T L E K P V D L L A G L P S S K D H Q S G R L V I
G P D Q K I Y Y T I G D Q G R N Q L A Y L F L P N
Q A Q H T P T Q Q E L N G K D Y H T Y M G K V L R
L N L D G S I P K D N P S F N G V V S H I Y T L G
H R N P Q G L A F T P N G K L L Q S E Q G P N S D
D E I N L I V K G G N Y G W P N V A G Y K D D S G
Y A Y A N Y S A A A N K T I K D L A Q N G V K V A
A G V P V T K E S E W T G K N F V P P L K T L Y T
V Q D T Y N Y N D P T C G E M T Y I C W P T V A P
S S A Y V Y K G G K K A I T G W E N T L L V P S L
K R G V I F R I K L D P T Y S T T Y D D A V P M F
K S N N R Y R D V I A S P D G N V L Y V L T D T A
G N V Q K D D G S V T N T L E N P G S L I K F T Y
K A K
Mature protein (SEQ ID NO: 1)
D V P L I P S Q F A K A K S E N F D K K V I L S N
L N K P H A L L W G P D N Q I W L T E R A T G K I
L R V N P E S G S V K T V F Q V P E I V N D A D G
Q N G L L G F A F H P D F K N N P Y I Y I S G T F
K N P K S T D K E L P N Q T I I R R Y T Y N K S T
D T L E K P V D L L A G L P S S K D H Q S G R L V
I G P D Q K I Y Y T I G D Q G R N Q L A Y L F L P
N Q A Q H T P T Q Q E L N G K D Y H T Y M G K V L
R L N L D G S I P K D N P S F N G V V S H I Y T L
G H R N P Q G L A F T P N G K L L Q S E Q G P N S
D D E I N L I V K G G N Y G W P N V A G Y K D D S
G Y A Y A N Y S A A A N K T I K D L A Q N G V K V
A A G V P V T K E S E W T G K N F V P P L K T L Y
T V Q D T Y N Y N D P T C G E M T Y I C W P T V A
P S S A Y V Y K G G K K A I T G W E N T L L V P S
L K R G V I F R I K L D P T Y S T T Y D D A V P M
F K S N N R Y R D V I A S P D G N V L Y V L T D T
A G N V Q K D D G S V T N T L E N P G S L I K F T
Y K A K

Generally, residue numbering will be for the mature protein (SEQ ID NO:1). Amino acids 25-478 of SEQ ID NO:50 are residues 1-454 of the mature protein.

Our choice fell on the loop connecting strands A and B of the β-sheet 3 (FIG. 1A, B). Beginning of the strand A harbors His144 that acts as the general base that abstracts a proton from the glucose O1 atom^{[9]}. As His144 is critical for catalysis we conjectured that its dislocation via torsion introduced by separation of strands A and B would lead to change of GDH catalytic activity. We decided to test this idea by incorporating into the loop connecting strands 3A and 3B a protein domain known to undergo large conformational changes upon ligand binding (FIG. 1B).

The loop faces away from the structure and the second subunit in homodimer and reducing the likelihood of steric clashes. As an insertion domain we chose Calmodulin (CaM)—a 17 kDa protein, which plays a major role in the transmission of calcium signals to target proteins in eukaryotes^{[10]}. The binding of Ca²⁺ to the four EF hands of CaM results in large conformational changes that open up a peptide binding pocket within each of the two lobes of CaM. This feature of CaM has been repeatedly exploited for construction of genetically encodable Ca²⁺ sensors based on either spectral changes or FRET intensity of fluorescent proteins or activity of b-lactomase^{[11][12]}. Based on the available structural information for both GDH and CaM^{[10]} we designed a chimeric protein where residues of mouse CaM 12-67 were inserted between the residues 153 and 155 of PQQ-GDH. In order to reduce structural tension and clashes we also introduced a GSGS linker at N-terminal of Calmodulin and a Gly linker at C-terminal of Calmodulin in the junction site. The resulting protein was periplastically produced in E. coli in recombinant from and purified to homogeneity by Ni-NTA affinity chromatography. We then used an established colorimetric assay to analyse the activity of GDH-CaM chimeric proteins^{[13]}. As can be seen in FIG. 2 in the absence of Ca²⁺ ions the CaM-GDH displayed virtually no enzymatic activity. Addition of CaCl₂ resulted in dose-dependent activation of the chimeric protein while having only limited effect on the wild-type enzyme. The activation was reversible as addition of the Ca²⁺ chelator EDTA returned the enzyme to the ground state (FIG. 2A). The signal change under the chosen experimental conditions was 30-fold. Analysis of Ca²⁺ titration data revealed a non-liner response to Ca²⁺ concentration which is has been described for CaM previously and is reflective of cooperative Ca²⁺ binding^{[14]}.

The fit of the data demonstrated that the developed sensor has Ca²⁺ affinity of 600 nM and displays the largest signal change between 400 and 500 nM. While the Kd of 600 nM reflects the affinity of CaM for ionized in the intracellular environment, the extracellular concentration of this ion is much higher. The bodily fluids such as blood, urine and saliva feature Ca²⁺ concentrations between 1 and 2 mM^{[15]}. The ability to rapidly assess these parameters is important in clinics since the deviation from the standard concentration is often reflective of chronic pathological states such as endocrine disorders, osteoporosis and cancer or acute states such as sepsis and acute renal failure ^{[15]}. Therefore rapid and accurate test for ionized calcium would be quite valuable for point of care (POC) diagnostics. Although in principle one could simply dilute the biological sample till Ca²⁺ concentration falls into sensor's response range this would significantly reduce its attractiveness for POC applications. Instead we tested whether we could buffer Ca²⁺ concentration to bring its free concentration into sensors response range. To this end we repeated our experiments in the presence of well characterized Ca²⁺ chelator 1,2-Bis(2-Aminophenoxy)ethane-N,N,N′,N′-tetraacetic acid (BAPTA). Addition of 1.1 mM of BAPTA to the sample allowed us to shift the optimum of the sensor's response into in the range of the physiological Ca²⁺ concentration in a blood sample (FIG. 2C). We also tested, whether the presence of Mg²⁺ would affect the response of the Ca²⁺ biosensor. The results shown in the FIG. 2C demonstrated that activation our chimeric GDH by Ca²⁺ is not influenced by the presence of Mg²⁺. This results support the notion that the developed GDH-Cam chimeric protein is suitable for construction of the POC biosensors.

The feature that enabled the development of inexpensive glucometer strips was the ability of GDH to retain its activity following de- and re-hydration. We tested the ability of our chimeric protein to retain the activity after drying and re-hydration and confirmed that similar to the wild type GDH the chimeric protein retained its activity upon rehydration. To further analyze the utility of the developed biosensors for analytical and diagnostic applications with sought to create a Ca²⁺ meter on the basis of a standard glucometer. To this end we stripped the standard Akku-Chek electrode of the enzyme mixture and replaced it with a mixture of GDH-Cam chimeric protein, glucose and PMS mediator. We demonstrated that the sensory electrode was activated by the Ca²⁺ ions present in human saliva and the current generated by the biosensor could be detected using a standard glucometer (FIG. 13).

Referring now to FIGS. 4-9 and 15 (SEQ ID NOS:2-10), an embodiment of an engineered, “dead” or catalytically inactive GDH enzyme is shown. The original idea was to split GDH into two portions or fragments: first portion or fragment is GDH(1-153AA); second portion or fragment is GDH(155-454AA). Binding moieties may then be coupled to each portion for detecting target molecules. For example, for the rapamycin sensor, we attached FRB to GDH(1-153AA) and FKBP to GDH(154-454AA).

However, only protein GDH(1-153AA)-FRB could be purified from E coli, not FKBP-GDH(154-454AA). Therefore, a different approach was taken to produce a construct which could be purified from E coli: GDH(1-153AA, Q76A, D143A, H144A)-TVMV cleavage site-FKBP-GDH(155-454AA).

H144 of GDH is the key catalytic residue of GDH. Based on initial experimental data, only a triple mutation (Q76A, D143A, H144A) made this GDH variant inactive. After cleavage by TVMV, the FKBP-GDH(154-454AA) was available for reconstitution of whole, catalytically active GDH through the binding of FRB and FKBP to Rapamycin.

In addition, even if we split GDH into two parts, they still can reconstitute by themselves. The affinity (K_{d}) for the self-reconstitution is around 50-100 nM. Therefore, for current assay conditions, we set up the concentration of two parts of GDH lower than 100 nM, in order to have low extent of self-reconstitution (results in low activity background). Another approach is to perform the assay with a high excess of GDH(1-153AA, Q76A, D143A, H144A) to prevent the self-reconstitution of GDH(1-153AA)-FRB with FKBP-GDH(155-454AA).

In summary we present here the first example of successful structure-guided engineering of Ca²⁺ gated electrochemical sensor. Our approach takes the departure of reported approach for specificity engineering using modifications of the catalytic site or prosthetic group modifications^{[8][16]}. Instead we identified an insertion site in the middle of GDH molecule that is close to catalytic center and is tolerant to insertion of large autonomously folding protein domains. The domain insertion strategy has been previously employed on several occasions to successfully to create artificial allosterically regulated enzymes^{[17][18]} but have never been applied to electrochemically active enzymes. The described approach changes the operational mode of the biosensor where it catalytic rate no longer limited by the glucose concentration but is gated by the chosen analyte. The high turnover rate of GDH makes it a very attractive chassis for engineering of other types of sensors. The identified insertion site in GDH now can be exploited for insertion of other sensory domains. While this process is largely empirical and involves significant amount of optimization the available data suggest that once insertion sites in protein are identified they can be exploited for insertion of multiple domains^{[12]}. Given the ubiquitous use of GDH-based glucose sensors and availability of end point and continues measurement devices our approach promises rapid path for introduction of Ca²⁺ sensors into clinical practice.

We also developed a generic biosensor architecture based on inactive engineered GDH fragments that form an active enzyme upon exposure to an chosen analyte. We demonstrated that by exchanging ligand recognition synthetic electrochemical receptors to proteins, small molecules and enzymatic activities can be constructed.

### Two Component Receptor Architecture Based on Split GDH

Analysis of the properties of the previously reported Calmodulin-GDH biosensor Ca2+ biosensor convinced us that GDH was an excellent building block for construction of synthetic electrochemical receptors. Its high physical stability combined with very high catalytic rate (K_{cat} 3860 per second) makes it an ideal actuator for direct interfacing with electronic devices. The observation that the loop connecting strands A and B of the β-sheet 3 could tolerate insertion of large domains prompted us to test whether the enzyme could be split at this position and used to construct a two component reconstitution system (FIG. 4). To this end we expressed N and C-terminal portions of the enzyme in E. coli in fusion with FRB and FKBP domains that form a complex in presence of rapamycin. While the FRB-GDH₁₋₁₅₃ could be produced and purified to homogeneity, the FKBP-GDH₁₅₅₋₄₅₄ formed inclusion bodies and could not be produced in the soluble form. This is not entirely surprising given the fact that the split exposes the hydrophobic core of the protein. Split proteins generally perform much better in vivo than in vitro owing to the compromised integrity and stability that to some extent can be compensated by the chaperon systems in vivo.

However, we found the encouragement in the fact that the N-terminal fragment of GDH was physically stable and apparently folded autonomously. We therefore conjectured that if the C-terminal fragment could be stabilised in solution we may be able to proceed with our original plan. To this end we constructed a variant of GDH with the TVMV cleavage site in the loop connecting strands A and B and carrying a FKBP domain on its C-terminus. We also mutated the catalytically important residues Gln₇₆, Asp₁₄₃ and His₁₄₄ in the active site to Ala rendering the fusion protein catalytically dead.

The resulting protein was produced recombinantly in soluble form and as expected displayed no detectable catalytic activity. When the fusion protein was mixed with the purified FRB-GDH₁₋₁₅₃ in the presence and in the absence of Rapamycin very little activity was recovered. We then digested the protease cleavage site containing loop connecting N- and C-fragments of GDH and repeated the reconstitution experiment. As in previous case very little GDH activity was recovered. However when the system was exposed to rapamycin we observed rapid and dose dependent recovery of GDH activity indicating that the inactivated N-terminal fragment of GDH was displaced by the FRB-fused GDH₁₋₁₅₃ fragment (FIGS. 4 and 5A). We titrated the reaction with increased concentrations of rapamycin and fitted the observed rates to a quadratic equation. The obtained value of 11 nM corresponded well with the previously published values of 12 nM by Banaszynski et al (J Am Chem Soc 2005, 127: 4715-21). Importantly, the reaction was specific as an excess of the related immunosuppressant compounds such as FK506 and cyclosporine did not activate the biosensor to an appreciable degree.

### Testing the Generic Nature of the Developed Biosensor Architecture.

We next set to test whether the developed architecture was sufficiently generic and could be expanded onto other biomarkers. As rapamycin (Sirolimus) belongs to the class of macrocyclic immunosuppressants together with cyclosporine and FK506 (Tacrolimus) that do not cross react with the developed biosensor we wondered if similar biosensors could be developed for these drugs as well. We were additionally motivated by the fact that immunosuppressants have very narrow therapeutic window and currently no point of care test for them exists.

We analysed the available co-crystal structures of cyclosporine with calcinurin A and B and peptidyl-prolyl cis-trans isomerase (PDB: 1MF8) and FK506 in complex with calcinurin A and B and FKBP (PDB: 1TCO). The topology of the complex allowed us to design fusion proteins of GDH fragments that were expected to come into molecular proximity when the complex assembly is induced by a ligand. The fusion proteins were produced in recombinant and purified form (Table 1). The developed biosensors responded to the cognate drug in a dose dependent manner (FIGS. 5B and C) and displayed no detectable cross reactivity even when the non-cognate drug was present at high concentration. These results demonstrated that the developed biosensor architecture could be rapidly adopted to detection of xenobiotics with known targets.

In the next step we wanted to test whether the developed biosensor architecture could be applied to the detection of protein biomarkers. As the first example we chose salivary α amylase that is commonly used as human stress biomarker. We analysed the available crystal structures of alpha amylase and identified three crystal structures of porcine α-amylase bound to VHH domains (PDB: 1KXQ, 1KXT, 1KXV and 1BVN). As human and porcine alpha amylase are 97% sequence identical we expected that the VHH domains will be able to recognise human salivary α-amylase. Based on structural analysis, we assumed that VHH domains extracted from PDB: 1KXV and PDB: 1BVN would bind to human salivary alpha amylase non-competitively. Therefor we constructed fusion proteins with GDH₁₋₁₅₃-VHH_{1KXV} and GDH inactive mutant with insertion of VHH_{1BVN} and produced them in recombinant form. Addition of human salivary alpha amylase to the mixture of both recombinant proteins resulted in a dose dependant increase in GDH activity that decreased at higher concentration reflecting formation of alpha amylase complexes with only one fusion protein bound to it. These results demonstrate that the developed sensory architecture could be used for detection of protein biomarkers (FIG. 6).

Finally we wanted to establish whether the developed sensor architecture could be used to measure enzymatic activities rather than chemical entities. As a test example we chose proteases that constitute the largest class of proteins on earth. In order to connect the protease activity to reconstitution of GDH from fragments we created an autoinhibited version of SH3 domain in which an SH3 domain binding peptide was linked to SH3 domain via protease—digestible linker (FIG. 8). In this configuration SH3 domain is protected from the binding of an external SH3 domain binding peptide as long as the linker connecting the domain and its ligand is intact. We fused the autoinhibited SH3 module to GDH₁₋₁₅₃ C-terminally while SH3 peptide was inserted into inactive full-length GDH with protease cleavage site at its N-terminal. In this design we placed a Factor Xa cleavage site between the SH3 domain and its ligand, and as well as between inactive N-terminal of GDH₁₋₁₅₃ and SH3 binding peptide. When the solution of the constructs produced in the recombinant form were mixed together only little GDH activity could be detected suggesting that SH3 peptide was not able to drive enzyme's reconstitution. However addition of Factor Xa resulted in time dependent increase of GDH activity indicating that proteolytic removal of the auto-inhibitory peptide enables binding of the SH3 peptide in trans and reconstitution of the active complex. In order to demonstrate the universal nature of the protease biosensor we replaced the factor Xa cleavage site with that of thrombin. We repeated the same experiment using a thrombin cleavage site and thrombin protease and observed a robust activation of the biosensor in the presence of the protease.

When comparing the response rate of the protease biosensor to that of immunosuppressant and amylase we noticed that the former was activated significantly slower. We suspected that this was due to the low Km of thrombin resulting in comparatively slow rates of linker digestion. We conjectured that introducing an extra thrombin binding motif “KTAPPFDFEAIPEEYL” (SEQ ID NO: 39) into the linker would accelerate the overall cleavage reaction by reducing the complete dissociation of non-productive protease:peptide complex. Indeed incorporation of the two copies of the thrombin substrate sequence and extra binding motif increased both the response rate and the sensitivity of the biosensors (FIG. 8C). Similarly in case of the biosensor of factor Xa introduction of two additional cleavage sites into the linker connecting the SH3 and SH3 binding domain resulted in increase of sensitivity and the response rate (FIGS. 8 E and F).

### Analysis of Stability of the Developed Biosensors for Construction of Sensory Electrodes.

The overwhelming success of the GDH based glucose monitors is at least in part due to the high stability of the enzyme that allows dehydration of the biosensor on the electrode and its long term storage at ambient temperatures. To test whether the engineered versions of GDH could be desiccated and rehydrated in functional form we incubated dried rapamycin biosensor at different temperatures for up to 4 hours. The data shows that no activity was lost up to 40 degree, and only little reduction in activity was observed up to 50 degree. We also left dried rapamycin biosensor at room temperature up to 14 days with no detectable change in activity indicating that they are suitable for construction of sensory electrodes. Next we tested the performance of the biosensors in chronoamperometric measurements using a commercial potentiostate. Chonoamperometric measurements were carried out using an Autolab PGSTAT204 potentiostat (Eco Chemie) interfaced to DropSens disposable screen printed gold electrodes (Cat#DRP-C220BT). A fresh sensor was used for each measurement. Reactions contained: 22.5 nM AMY-1, 18.7 nM AMY-2 PQQ, 3.7 nM TVMV, 1.0 mM 1-methoxy-5-methylphenazinium methyl sulfate, 50 mM glucose, 2.0 mM MgCl₂, 50 uM CaCl₂ and 0, 2, 4, 6, 8, 10, 15, 20, 30, 40 50, 100, 200, 300, 400, 600, 800 or 1000 nM human salivary alpha amylase in 45 μl total volume at pH 7.4 PBS. Reactions were started with the addition of 1 mM 1-methoxy-5-methylphenazinium methyl sulfate and 50 mM glucose and incubated at room temperature for 30 seconds before being pipetted onto the electrode surface. After a wait time of 180 seconds, chronoamperometry was carried out for 5 s at +0.4 V versus the imbedded silver strip on the screen printed electrode, with data generally reported as current at the 5 s time point versus amylase concentration.

### Engineering of Autoinhibited GDH Enzymes

We further engineered GDH enzymes including inhibitory moieties which autoinhibit catalytic activity enzyme of the enzyme, which can then be cleaved from the enzyme by a protease to reconstitute activity. The protease cleavage event can also be tied to a binding interaction between different components of a biosensor, dependent on presence of a target molecule. Autoinhibited enzymes of this type are shown in FIGS. 3A-E together with data showing activation of the enzyme and detection of a target molecule. Mutations were also made to the GDH enzyme to provide for improved anchoring of inhibitory peptides to the active site, and inhibitory peptides providing for inhibition of activity in these mutants were further identified. The resulting autoinhibited GDH module provides a generic platform for protease activity detection where the specificity of the biosensor can be changed by replacing the protease cleavage site with the recognition site for the respective protease.

Availability of the autoinhibited (AI) GDH module enables construction of different receptor architectures such as two component receptor where AI-GDH module is brought into proximity of a protease by action of operably connected binding domains scaffolded through interaction with a ligand (FIG. 3E). This is exemplified by a rapamycin receptor constructed on the based of the developed AI-GDH module. Further, the same AI-GDH unit could be integrated into the reversible receptor architecture exemplified in the FIG. 3G. Furthermore, reversible analyte-mediated activation of AI-GDH module can be achieved by integrating an analyte binding domain that undergoes a conformational change upon ligand binding into the linker connecting GDH with AI (FIG. 3H) Inhibitory moieties used in some of the above experiments were also identified through an in vitro screening assay. In more detail, autoinhibited GDH modules from independent functional protein domains (e.g. reporter enzymes, corresponding active site specific binders and analyte specific binding receptors) can be engineered using a medium throughput assay in 96 well plates directly in the supernatant of E. coli cell cultures. To this end, a DNA library of putative active site specific binders (that have previously been identified by means of phage display or related screening and selection procedure) is first inserted C-terminal of the reporter enzyme GDH separated by a flexible linker and a cleavage site for TVMV which act as an analyte specific receptor. The resulting fusion protein is cloned under the control of an IPTG inducible promoter and PelB leader peptide for periplasmic expression in the backbone of a pET-28 vector which confers kanamycin resistance. Following transformation into E. coli BL21 RIL, cells are plated on agar plates in the presence of kanamycin and grown overnight at 37° C. The following day, 200 μL LB medium supplemented with kanamycin and chloramphenicol is inoculated with individual colonies, and grown for 5-6 hours at 37° C. The expression of the putative autoinhibited GDH modules is then induced with 0.2 mM IPTG and left to express for 24 h at 18° C. In order to assay for autoinhibited GDH modules, the supernatant of the cell culture is isolated by centrifuging cells at 4,500 g for 10 min while the resulting supernatant (45 μL) is incubated in the presence and absence of TVMV protease (5 μL at 1 mg/mL protease) for 1 h, and GDH activity measured using 600 nM 2-6-dichlorophenyl-Indophenol (DCPIP), 600 nM phenazine methosulfate (PSF), 60 nM PQQ and 20 mM glucose buffered with 100 μM CaCl₂ and 20 mM K₂HPO₄ at pH 7.0. The reaction is monitored by measuring the decrease in absorbance at 620 nM.

This assay provides a medium-throughput in vitro screen to allow for processing of putative inhibitory moieties.

### CONCLUSION

Moving diagnostics and analytics from the specialised facilities to the site of action such as bedside or a farm is expected to bring benefits both to individuals and the society as a whole. Among those are informed decision making, end user participation and cost reduction. To date, only electrochemical biosensors such as the glucose monitors meet the performance and cost benchmarks for their wide deployment. The success of the glucose monitors relates to the nature of the condition, abundance of the analyte that simultaneously serves as a source of energy and remarkable stability of the biosensor.

Here we build on the highly advanced glucometer technology by re-engineering to its principle biosensor GDH into a generic biosensor architecture. By exploiting the remarkable biophysical stability of the enzyme we convert it into a two component signalling system where individual components are neither active nor capable of reassembly. This is achieved by creating a split enzyme with inactive fragment that serves both as an inhibitor of spontaneous activation and as a chaperone factor ensuring the correct folding and stability of the active half of the enzyme. This sets the developed architecture apart from the other split enzyme systems that often suffer from the inferior biophysical properties limiting their utility for in vitro applications.

Activation of the developed system is achieved through the analyte-mediated scaffolding of the fragments that results in the concentration driven replacement of the inactive N-terminal fragment with its active form. The kinetics of activation appears to be surprisingly rapid, indicating high off rate of the N- and C-terminal complex. In practical terms this translates in the rapid rate of system's response making it suitable for point of care applications. We used the developed basic architecture to construct a range of biosensors to small molecules, proteins and protease activities. The sensors demonstrated excellent response rate, sensitivity and selectivity confirming that the system is truly modular and can be adopted practically any analyte for which a binding domain can be found. In principle the system can be expanded to semisynthetic systems and used to detect association events mediated by small molecules, DNA, RNA and other types of biological, synthetic polymers and post translational modifications.

This has potentially important implications as the ubiquitous presence of the internet enabled personal electronic devices has long supported the idea of personal mobile analytic and diagnostic applications. However, miniaturisation of the standard analytic technologies so far was not able to deliver biosensing applications sufficiently small, inexpensive and portable to be easily hosted on the mobile devices. The approach presented here potentially allows to adopt the inexpensive and portable technology developed for glucose monitoring for potentially any analyte. The high catalytic rate of GDH and the resulting electron current simplify the detection of the molecular recognition events potentially enabling electrode miniaturisation and multiplexing.

TABLE 1
Protein components for drug sensors

| | | |
|---|---|---|
| Rapamycin | GDH₁₋₁₅₃-FRB-His₆ | GDH_{1-153 inactive mutant}-FKBP-GDH₁₅₅₋₄₅₄- |
| | | His₆ |
| FK506 | Co-expression of | GDH_{1-153 inactive mutant}-FKBP-GDH₁₅₅₋₄₅₄- |
| | GDH₁₋₁₅₃-Calcinurin B- | His₆ |
| | His₆ | |
| | His₆-Calcinurin A | |
| Cyclosporine A | Co-expression of | GDH_{1-153 inactive mutant}-GDH₁₅₅₋₄₅₄-CYPA- |
| | GDH₁₋₁₅₃-Calcinurin B- | His₆ |
| | His₆ | |
| | His₆-Calcinurin A | |
| Salivary alpha | GDH₁₋₁₅₃-VHH_{1KXV}-His₆ | GDH_{1-153 inactive mutant}-VHH_{1BVN}-GDH₁₅₅₋₄₅₄- |
| amylase | | His₆ |
| Thrombin | GDH₁₋₁₅₃-SH3-Thr-SH3 | GDH_{1-153 inactive mutant}-Thr-SH3 binding |
| sensor | bindig peptide-His₆ | peptide-GDH₁₅₅₋₄₅₄-His₆ |
| Factor Xa | GDH₁₋₁₅₃-SH3-FX-SH3 | GDH_{1-153 inactive mutant}-FX-SH3 binding |
| sensor | bindig peptide-His₆ | peptide-GDH₁₅₅₋₄₅₄-His₆ |

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

### REFERENCES

[1] A. Turner, Trends Biotechnol. 2013, 31, 119-20.
[2] N. J. Ronkainen, H. B. Halsall, W. R. Heineman, Chem. Soc. Rev. 2010, 39, 1747-63.
[3] S. D. Psoma, P. D. van der Wal, O. Frey, N. F. de Rooij, A. P. F. Turner, Biosens. Bioelectron. 2010, 26, 1582-7.
[4] E.-H. Yoo, S.-Y. Lee, Sensors (Basel). 2010, 10, 4558-76.
[5] V. Scognamiglio, Biosens. Bioelectron. 2013, 47, 12-25.
[6] J. Hu, X.-E. Zhang, J. Diabetes Sci. Technol. 2011, 5, 1294-9.
[7] A. Oubrie, Biochim. Biophys. Acta—Proteins Proteomics 2003, 1647, 143-151.
[8] S. Igarashi, J. Okuda, K. Ikebukuro, K. Sode, Arch. Biochem. Biophys. 2004, 428, 52-63.
[9] a Oubrie, H. J. Rozeboom, K. H. Kalk, J. a Duine, B. W. Dijkstra, J. Mol. Biol. 1999, 289, 319-33.
[10] P. Kursula, Amino Acids 2014, 46, 2295-304.
[11] V. Pérez Koldenkova, T. Nagai, Biochim. Biophys. Acta 2013, 1833, 1787-97.
[12] G. E. Meister, N. S. Joshi, Chembiochem 2013, 14, 1460-7.
[13] K. Abe, W. Yoshida, K. Terada, Y. Yagi-Ishii, S. Ferri, K. Ikebukuro, K. Sode, Int. J. Mol. Sci. 2013, 14, 23244-56.
[14] N. V Valeyev, D. G. Bates, P. Heslop-Harrison, I. Postlethwaite, N. V Kotov, BMC Syst. Biol. 2008, 2, 48.
[15] W. G. Robertson, R. W. Marshall, Crit. Rev. Clin. Lab. Sci. 1981, 15, 85-125.
[16] E. Katz, A. Riklin, V. Heleg-shabtai, I. Willner, A. F. Bu, 1999, 385, 45-58.
[17] G. Guntas, T. J. Mansell, J. R. Kim, M. Ostermeier, Proc Natl Acad Sci USA 2005, 102, 11224-11229.
[18] A. V Karginov, F. Ding, P. Kota, N. V Dokholyan, K. M. Hahn, Nat. Biotechnol. 2010, 28, 743-7.
[19] Olsthoorn & Duine, Arch. Biochem. Biophys. 1996, 336, 42-48
[20] Yu et al., Engineering in Life Sciences. 2012, 12, 574-582
[21] Frey & Gorlich, J. Chromatography A. 2014 1337, 95-105

1.-45. (canceled)

46. A biosensor comprising:
(i) at least one amino acid sequence of an enzyme capable of reacting with a substrate molecule when in a catalytically active state to produce one or more electrons; and at least one heterologous, sensor amino acid sequence that releasably maintains the enzyme in a catalytically inactive state, wherein the heterologous, sensor amino acid sequence is responsive to a target molecule to switch the amino acid sequence of the enzyme from the catalytically inactive state to said catalytically active state;
(ii) at least one amino acid sequence of an enzyme capable of reacting with a substrate molecule when in a catalytically active state to produce one or more electrons; and at least one other amino acid sequence of said enzyme which is engineered to releasably maintain the enzyme in a catalytically inactive state, wherein the biosensor is responsive to a target molecule to switch the amino acid sequence of the enzyme from the catalytically inactive state to said catalytically active state; or
(iii) at least one amino acid sequence of an enzyme capable of reacting with a substrate molecule when in a catalytically active state to produce one or more electrons; a binding moiety capable of binding a target molecule; and at least one enzyme inhibitor which is capable of interacting with the binding moiety in the absence of the target molecule to thereby inhibit the enzyme; arranged so that the target molecule can release the interaction between said at least one enzyme inhibitor and the binding moiety to thereby release inhibition of the enzyme by the inhibitor and switch the amino acid sequence of the enzyme from a catalytically inactive state to said catalytically active state.

47. The biosensor of claim 1(i), wherein the at least one enzyme amino acid sequence and said at least one heterologous, sensor amino acid sequence are present in, or form at least part of a single, contiguous amino acid sequence, optionally wherein said at least one heterologous, sensor amino acid sequence is an insert in said at least one enzyme amino acid sequence, to thereby facilitate switching the enzyme amino acid sequence between said catalytically inactive and said catalytically active state.

48. The biosensor of claim 1(i), wherein the heterologous, sensor amino acid sequence:
(a) binds said target molecule to thereby switch the amino acid sequence of the enzyme from the catalytically inactive state to said catalytically active state; and/or
(b) is an amino acid sequence of a calcium-binding protein, or a fragment thereof, optionally wherein the calcium-binding protein is calmodulin.

49. The biosensor of claim 1(ii), wherein said at least one other amino acid sequence of said enzyme is engineered to comprise one or more amino acid sequence mutations; optionally wherein said at least one amino acid sequence of the enzyme capable of reacting with a substrate molecule when in a catalytically active state to produce one or more electrons and said at least one other amino acid sequence of said enzyme engineered to releasably maintain the enzyme in a catalytically inactive state, non-covalently interact.

50. The biosensor of claim 1(ii), wherein said at least one other amino acid sequence of said enzyme is engineered to comprise one or more amino acid sequence mutations and wherein said at least one amino acid sequence of the enzyme capable of reacting with a substrate molecule when in a catalytically active state to produce one or more electrons and said at least one other amino acid sequence of said enzyme engineered to releasably maintain the enzyme in a catalytically inactive state, non-covalently interact and wherein said biosensor further comprises yet another amino acid sequence of said enzyme which is capable of replacing said at least one other amino acid sequence of said enzyme engineered to releasably maintain the enzyme in a catalytically inactive state, optionally wherein replacement restores the catalytic activity of the enzyme by non-covalently combining said yet another amino acid sequence of said enzyme with said at least one amino acid sequence of the enzyme capable of reacting with a substrate to form a functional, catalytically active enzyme, optionally wherein:
(a) said yet another amino acid sequence of said enzyme and said at least one amino acid sequence of the enzyme capable of reacting with a substrate comprise respective binding moieties that can interact by binding a target molecule, to facilitate the replacement of the engineered amino acid sequence by said yet another amino acid sequence; or
(b) said engineered amino acid sequence of said enzyme and said at least one amino acid sequence of the enzyme capable of reacting with a substrate comprise respective binding moieties which initially interact, which interaction is subsequently disrupted by one or the other of the binding moieties binding a target molecule to facilitate the replacement of the engineered amino acid sequence by said yet another amino acid sequence.

51. The biosensor of claim 1(ii), wherein the at least one amino acid sequence of the enzyme represents a first fragment sequence of said enzyme and the at least one other amino acid sequence or the yet another amino acid sequence of said enzyme represents a said second fragment sequence of said enzyme, wherein said first and second fragment sequences are able to non-covalently interact to reconstitute a stable enzyme.

52. The biosensor of claim 1(ii), which is suitable for detecting a protease target molecule, comprising one or more protease cleavage sites in a said amino acid sequence of said enzyme, optionally wherein said amino acid sequence further comprises a sequence enhancing binding and/or cleavage efficiency of the protease, optionally wherein said at least one amino acid sequence of the enzyme capable of reacting with a substrate and said yet another amino acid sequence of the enzyme comprise respective binding moieties that can interact after protease cleavage of an inhibitor of binding between these.

53. The biosensor of claim 1(iii), comprising a first component comprising: at least one amino acid sequence of an enzyme capable of reacting with a substrate molecule when in a catalytically active state to produce one or more electrons; an inhibitor of said enzyme linked or coupled to the enzyme by one or more protease cleavage sites; and a first component binding moiety; a second component comprising a second component binding moiety capable of binding the first component binding moiety; a protease amino acid sequence; and another second component binding moiety capable of binding a target molecule; and a third component comprising a third component binding moiety that can interact with said second component binding moieties in the absence of the target molecule; arranged so that said target molecule can displace binding between the third component binding moiety and said second component binding moieties to facilitate an interaction between said first component binding moiety and said second component binding moiety whereby the protease cleaves the protease cleavage site(s) to remove inhibition of the enzyme by the inhibitor and thereby switch the enzyme from a catalytically inactive state to a catalytically active state, optionally further comprising a sequence enhancing binding and/or cleavage efficiency of the protease located proximally to said protease cleavage site; optionally, wherein the third component binding moiety comprises a first portion that can interact with one of said second component binding moieties and a second portion that can interact with another of said second component binding moieties in the absence of the target molecule, optionally wherein binding of the target molecule by said another of said second component binding moieties inhibits binding of the second portion of the third component.

54. The biosensor of claim 1, wherein said enzyme is an oxidoreductase enzyme, such as a glucose dehydrogenase enzyme.

55. An enzyme which is:
(i) a glucose dehydrogenase (GDH) enzyme comprising a heterologous, sensor amino acid sequence which is responsive to a target molecule, wherein binding of the target molecule acts to regulate catalytic activity of the enzyme; or
(ii) an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme.

56. The GDH enzyme of claim 55(i), wherein the heterologous, sensor amino acid sequence is an amino acid sequence of a calcium-binding protein, or a functional fragment thereof.

57. The oxidoreductase enzyme of claim 55(ii), comprising one or more protease cleavage sites, wherein cleavage of a said site by a protease displaces the inhibitory moiety to activate catalytic activity of the enzyme, optionally further comprising a sequence enhancing binding and/or cleavage efficiency of the protease.

58. The oxidoreductase enzyme of claim 55(ii), comprising a binding moiety capable of interacting with a respective binding moiety on a further molecule, wherein interaction between the binding moieties displaces the inhibitory moiety to activate catalytic activity of the enzyme, optionally comprising one or more protease cleavage sites, wherein the further molecule additionally comprises a protease and interaction between the binding moieties acts to bring the protease into proximity with a said site to cleave said site and displace the inhibitory moiety.

59. The oxidoreductase enzyme of claim 55(ii), which is a glucose dehydrogenase enzyme.

60. A polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme.

61. The polypeptide comprising a first fragment sequence of a GDH enzyme of claim 60, which is capable of reconstituting a stable catalytically active GDH enzyme with said polypeptide comprising a second fragment sequence of said enzyme.

62. The polypeptide comprising a first fragment sequence of a GDH enzyme of claim 60, which comprises one or more mutations which render the reconstituted stable GDH enzyme catalytically inactive.

63. The polypeptide comprising a first fragment sequence of a GDH enzyme of claim 60, which comprises a binding moiety capable of interacting with a respective binding moiety comprised in said polypeptide comprising a second fragment sequence of said enzyme, wherein the interaction between the binding moieties regulates catalytic activity of the reconstituted stable glucose dehydrogenase enzyme, optionally wherein the interaction between the binding moieties is regulated by binding of a target molecule;
optionally which further comprises a sequence inhibiting interaction of the respective binding moieties, and one or more protease cleavage sites, wherein cleavage by the protease provides for interaction between the binding moieties, optionally further comprising a sequence enhancing binding and/or cleavage efficiency of the protease.

64. A composition comprising the biosensor of claim 1, a glucose dehydrogenase enzyme comprising a heterologous, sensor amino acid sequence which is responsive to a target molecule, wherein binding of the target molecule acts to regulate catalytic activity of the enzyme; an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme; optionally further comprising a substrate molecule.

65. A kit comprising the biosensor of claim 1, a glucose dehydrogenase enzyme comprising a heterologous, sensor amino acid sequence which is responsive to a target molecule, wherein binding of the target molecule acts to regulate catalytic activity of the enzyme; an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme; optionally further comprising a substrate molecule.

66. A detection device comprising a cell or chamber comprising the biosensor of claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme.

67. An isolated nucleic acid encoding the biosensor of claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme.

68. A genetic construct comprising an isolated nucleic acid encoding the biosensor of claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme.

69. A host cell comprising a genetic construct comprising an isolated nucleic acid encoding the biosensor of claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme.

70. A method of detecting a target molecule, said method including the step of contacting the biosensor of claim 1, or a composition comprising the biosensor of claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme, with a sample to thereby determine the presence or absence of the target molecule in the sample, optionally for detecting a performance enhancing substance or an illicit drug in the mammal.

71. A method of diagnosing a disease or condition in an organism, said method including the step of contacting the biosensor claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme, with a biological sample obtained from the organism to thereby determine the presence or absence of a target molecule in the biological sample, determination of the presence or absence of the target molecule facilitating diagnosis of the disease or condition.

72. A method of producing a recombinant protein biosensor or a component thereof of claim 1, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme, said method including the step of producing the recombinant protein biosensor or component thereof or the GDH enzyme or oxidoreductase enzyme or polypeptide comprising a first or second fragment sequence of a GDH enzyme in a host cell comprising a genetic construct comprising an isolated nucleic acid encoding the biosensor of claim 1, or a component thereof, a glucose dehydrogenase enzyme comprising a heterologous, sensor amino acid sequence which is responsive to a target molecule, wherein binding of the target molecule acts to regulate catalytic activity of the enzyme, an oxidoreductase enzyme comprising an inhibitory moiety acting to prevent or reduce catalytic activity of the enzyme, wherein the inhibitory moiety can be displaced in the presence of one or more molecules to activate catalytic activity of the enzyme; or a polypeptide comprising a first fragment sequence of a glucose dehydrogenase (GDH) enzyme, which is capable of non-covalently interacting with a polypeptide comprising a second fragment sequence of said enzyme to reconstitute a stable GDH enzyme.


SEQUENCE LISTING (61 sequences)

SEQ ID NO: 1 - protein, 454 aa, Artificial Sequence
  Mature PQQ-GDH protein
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 2 - protein, 267 aa, Artificial Sequence
  Rapamycin sensor: Component1: GDH(1-153AA)-FRB
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGSGSGGLWHEMWHEGLEEASRLYFGERNVKGMFEVLEPLHAMMERGPQTLKETSFNQAYGRDLMEAQEWCRKYMKSGNVKDLTQAWDLYYHVFRRISGKLAAALEHHHHHH

SEQ ID NO: 3 - protein, 587 aa, Artificial Sequence
  Component2: GDH(1-153AA, Q76A, D143A,H144A)- TVMV cleavage site-FKBP-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGSGGETVRFQSGGSGSGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 4 - protein, 351 aa, Artificial Sequence
  FK506 sensor: Component1-1: GDH(1-153AA)- Calcineurin alpha subunit
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGGSGGSGSGGSGGGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDVKLAAALEHHHHHH

SEQ ID NO: 5 - protein, 411 aa, Artificial Sequence
  FK506 sensor: Component1-2: Calcineurin beta subunit
AHHHHHHSSGTSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARLVTAGLVLA

SEQ ID NO: 6 - protein, 587 aa, Artificial Sequence; identical to SEQ ID NO: 3
  FK506 sensor: Component 3: GDH(1-153AA, Q76A, D143A,H144A)-TVMV cleavage site-FKBP-GDH(155-454AA)

SEQ ID NO: 7 - protein, 351 aa, Artificial Sequence; identical to SEQ ID NO: 4
  Cyclosporin A sensor: Component1-1: GDH(1-153AA)-Calcineurin alpha subunit

SEQ ID NO: 8 - protein, 411 aa, Artificial Sequence; identical to SEQ ID NO: 5
  Cyclosporin A sensor: Component1-2: Calcineurin beta subunit

SEQ ID NO: 9 - protein, 661 aa, Artificial Sequence
  Cyclosporin A sensor: Component2: GDH(1-153AA, Q76A, D143A,H144A)-TVMV cleavage site-GDH(155-454AA)-Cyclophilin
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGSGETVRFQSGSGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGGSGGSGGSGGSGGSGGMVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLEKLAAALEHHHHHH

SEQ ID NO: 10 - protein, 403 aa, Artificial Sequence
  Inactive dead GDH(1-153AA, Q76A, D143A, H144A)-GST
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGSGGNTSSNSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPTNRWVSMADKLAAALEHHHHHH

SEQ ID NO: 11 - protein, 7 aa, Artificial Sequence
  TVMV cleavage sites
ETVRFQS

SEQ ID NO: 12 - skipped

SEQ ID NO: 13 - protein, 153 aa, Artificial Sequence
  GDH 1-153AA
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGP

SEQ ID NO: 14 - protein, 153 aa, Artificial Sequence
  GDH 1-153AA, Q76A, D143A,H144A
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGP

SEQ ID NO: 15 - protein, 303 aa, Artificial Sequence
  GDH 155-454AA
GSGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 16 - protein, 168 aa, Artificial Sequence
  Calcineurin alpha subunit
NEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 17 - protein, 165 aa, Artificial Sequence
  Cyclophilin
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 18 - protein, 106 aa, Artificial Sequence
  FKBP
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 19 - protein, 92 aa, Artificial Sequence
  FRB
LWHEMWHEGLEEASRLYFGERNVKGMFEVLEPLHAMMERGPQTLKETSFNQAYGRDLMEAQEWCRKYMKSGNVKDLTQAWDLYYHVFRRISG

SEQ ID NO: 20 - protein, 8 aa, Artificial Sequence
  Inhibitory peptide identified for wt GDH (1)
ADARYKSG

SEQ ID NO: 21 - protein, 8 aa, Artificial Sequence
  Inhibitory peptide identified for wt GDH (2)
IPLFAYAG

SEQ ID NO: 22 - protein, 8 aa, Artificial Sequence
  Inhibitory peptide identified for wt GDH (3)
LLAPPYWG

SEQ ID NO: 23 - protein, 8 aa, Artificial Sequence
  Inhibitory peptide identified for wt GDH (4)
HWNTVVSG

SEQ ID NO: 24 - protein, 8 aa, Artificial Sequence
  Inhibitory peptide identified for wt GDH (5)
GGMFLWPG

SEQ ID NO: 25 - protein, 8 aa, Artificial Sequence
  Inhibitory peptide identified for GDH-Tyr mutant (1)
KVWIVSTG

SEQ ID NO: 26 - protein, 8 aa, Artificial Sequence
  Inhibitory peptide identified for GDH-Tyr mutant (2)
KVWIVSVG

SEQ ID NO: 27 - protein, 8 aa, Artificial Sequence
  Inhibitory peptide identified for GDH-Tyr mutant (3)
KVWVLEQG

SEQ ID NO: 28 - protein, 8 aa, Artificial Sequence
  Inhibitory peptide identified for GDH-Tyr mutant (4)
KVWVLNSG

SEQ ID NO: 29 - protein, 8 aa, Artificial Sequence
  Inhibitory peptide identified for GDH-Tyr mutant (5)
SHDIHYMG

SEQ ID NO: 30 - protein, 8 aa, Artificial Sequence
  Inhibitory peptide identified for GDH-Tyr mutant (6)
ADARYKSG

SEQ ID NO: 31 - protein, 460 aa, Artificial Sequence
  GDH-Tyr mutant sequence
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGSSSYSCWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 32 - protein, 496 aa, Artificial Sequence
  AI-GDH-Tyr construct 1
KVWVLEQGGLVPRGVGGGKTAPPFDFEAIPEEYLGGDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGSSSYSCWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 33 - protein, 496 aa, Artificial Sequence
  AI-GDH-Tyr construct 2
SHDIHYMGGLVPRGVGGGKTAPPFDFEAIPEEYLGGDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGSSSYSCWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 34 - protein, 6 aa, Artificial Sequence
  Thrombin cleavage site
LVPRGV

SEQ ID NO: 35 - protein, 4 aa, Artificial Sequence
  Factor Xa cleavage site
IEGR

SEQ ID NO: 36 - protein, 4 aa, Artificial Sequence
  Factor Xa cleavage site
IDGR

SEQ ID NO: 37 - protein, 57 aa, Artificial Sequence
  SH3 Scaffolding domain
AEYVRALFDFNGNDEEDLPFKKGDILRIRDKPEEQWWNAEDSEGKRGMIPVPYVEKY

SEQ ID NO: 38 - protein, 11 aa, Artificial Sequence
  SH3 ligand (SH3L)
PPPPLPPKRRR

SEQ ID NO: 39 - protein, 16 aa, Artificial Sequence
  Thrombin high affinity site
KTAPPFDFEAIPEEYL

SEQ ID NO: 40 - protein, 264 aa, Artificial Sequence
  Component1: GDH(1-153AA)-SH3-Thrombin site-SH3L
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGGSGGAEYVRALFDFNGNDEEDLPFKKGDILRIRDKPEEQWWNAEDSEGKRGMIPVPYVEKYGSGGSGGLVPRGVGGSGGSGPPPPLPPKRRRGGKLAAALEHHHHHH

SEQ ID NO: 41 - protein, 488 aa, Artificial Sequence
  Component2: GDH(1-153AA, Q76A, D143A,H144A)- Thrombin site-SH3L-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGLVPRGVGGPPPPLPPKRRRGGSGGSGGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 42 - protein, 283 aa, Artificial Sequence
  Component1: GDH(1-153AA)-SH3-Thrombin high affinity site-SH3L
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGGSGGAEYVRALFDFNGNDEEDLPFKKGDILRIRDKPEEQWWNAEDSEGKRGMIPVPYVEKYGSGGSGGLVPRGVGGGKTAPPFDFEAIPEEYLGGSGGSGPPPPLPPKRRRGGKLAAALEHHHHHH

SEQ ID NO: 43 - protein, 507 aa, Artificial Sequence
  Component2: GDH(1-153AA, Q76A, D143A,H144A)- Thrombin high affinity site-SH3L-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGLVPRGVGGGKTAPPFDFEAIPEEYLGGPPPPLPPKRRRGGSGGSGGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 44 - protein, 313 aa, Artificial Sequence
  Component1: GDH(1-153AA)-SH3-two Thrombin high affinity site-SH3L
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGGSGGAEYVRALFDFNGNDEEDLPFKKGDILRIRDKPEEQWWNAEDSEGKRGMIPVPYVEKYGSGGSGGLVPRGVGGGKTAPPFDFEAIPEEYLGSGLVPRGVGGGKTAPPFDFEAIPEEYLGGGGSGGSGPPPPLPPKRRRGGKLAAALEHHHHHH

SEQ ID NO: 45 - protein, 535 aa, Artificial Sequence
  Component2: GDH(1-153AA, Q76A, D143A,H144A)-two Thrombin high affinity site-SH3L-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGLVPRGVGGGKTAPPFDFEAIPEEYLGSGLVPRGVGGGKTAPPFDFEAIPEEYLGGPPPPLPPKRRRGGSGGSGGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 46 - protein, 264 aa, Artificial Sequence
  Component1: GDH(1-153AA)-SH3-Factor Xa site-SH3L
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGGSGGAEYVRALFDFNGNDEEDLPFKKGDILRIRDKPEEQWWNAEDSEGKRGMIPVPYVEKYGGSGGSGGIEGRGGSGGSGGPPPPLPPKRRRGGKLAAALEHHHHHH

SEQ ID NO: 47 - protein, 488 aa, Artificial Sequence
  Component2: GDH(1-153AA, Q76A, D143A,H144A)-one Factor Xa site-SH3L-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGIEGRGGSGPPPPLPPKRRRGGSGGSGGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 48 - protein, 274 aa, Artificial Sequence
  Component1: GDH(1-153AA)-SH3-three Factor Xa site-SH3L
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGGSGGSGGAEYVRALFDFNGNDEEDLPFKKGDILRIRDKPEEQWWNAEDSEGKRGMIPVPYVEKYGGSGGSGGIEGRGIDGRGIEGRGGSGGSGGPPPPLPPKRRRGGKLAAALEHHHHHH

SEQ ID NO: 49 - protein, 498 aa, Artificial Sequence
  Component2: GDH(1-153AA, Q76A, D143A,H144A)- three Factor Xa site-SH3L-GDH(155-454AA)
DVPLIPSQFAKAKSENFDKKVILSNLNKPHALLWGPDNQIWLTERATGKILRVNPESGSVKTVFQVPEIVNDADGANGLLGFAFHPDFKNNPYIYISGTFKNPKSTDKELPNQTIIRRYTYNKSTDTLEKPVDLLAGLPSSKAAQSGRLVIGPGGIEGRGIDGRGIEGRGGSGPPPPLPPKRRRGGSGGSGGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 50 - protein, 478 aa, Artificial Sequence
  PQQ-GDH protein sequence before cleavage of signal sequence
MNKHLLAKIALLGAAQLVTLSAFADVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 51 - protein, 12 aa, Artificial Sequence
  Inhibitory peptide
NSTHHHHFATIW

SEQ ID NO: 52 - protein, 216 aa, Artificial Sequence
  Affinity Clamp
KALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWE

SEQ ID NO: 53 - protein, 608 aa, Artificial Sequence
  Calcium biosensor (PQQ-GDH with CaM insertion between residues 153 and 155)
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPGADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 54 - protein, 754 aa, Artificial Sequence
  GDH[TVMV]-VH[B6]-FKBP12
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGGSGGSGGSGGETVRFQSGGSGGTGGSSGSGGSGGAMAEVQLLESGGGLVQPGGSLRLSCAASGFRFSDEDMGWVRQAPGKGLEWVSAIASHGDSTYYADSVKGRFTISRDNSKNTLYLQMNSLRAEDTAVYYCAKTVDGFSRITFDYWGQGTLVTVSSAAAGGSGGTSGGSGGSGGGSGGSGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGTGHHHHHH

SEQ ID NO: 55 - protein, 127 aa, Artificial Sequence
  VH[B6]
AMAEVQLLESGGGLVQPGGSLRLSCAASGFRFSDEDMGWVRQAPGKGLEWVSAIASHGDSTYYADSVKGRFTISRDNSKNTLYLQMNSLRAEDTAVYYCAKTVDGFSRITFDYWGQGTLVTVSSAAA

SEQ ID NO: 56 - protein, 7 aa, Artificial Sequence
  Inhibitory peptide identified by phage display Fig 3B
SHDIHYM

SEQ ID NO: 57 - protein, 4 aa, Artificial Sequence
  GSGS linker
GSGS

SEQ ID NO: 58 - protein, 46 aa, Artificial Sequence
  cross-binder, a calmodulin binding peptide
GVMPREETDSKTASPWKSARLMVHTVATFNSIKELNERWRSLQQLA

SEQ ID NO: 59 - protein, 4 aa, Artificial Sequence
  Positions 340-344 of SEQ ID NO: 1
EMTY

SEQ ID NO: 60 - protein, 5 aa, Artificial Sequence
  Positions 340-344 of SEQ ID NO: 1
EMTYI

SEQ ID NO: 61 - protein, 5 aa, Artificial Sequence
  Variant sequence
SSSYS